(19) United States
(12) Patent Application Publication
(10) Pub. No.: US 2022/0071730 A1
(43) Pub. Date: Mar. 10, 2022

(54) POSITION-SENSITIVE MAGNETICALLY-SENSITIVE TAGS FOR SURGICAL PROCEDURES

(71) Applicant: Tychon Technologies, Inc., Los Angeles, CA (US)

(72) Inventors: John Kelly, Los Angeles, CA (US); Azita Emami, Pasadena, CA (US); Mikhail Georgievich Shapiro, Los Angeles, CA (US)

(21) Appl. No.: 17/447,241

(22) Filed: Sep. 9, 2021

Related U.S. Application Data
(60) Provisional application No. 63/075,980, filed on Sep. 9, 2020.

(51) Int. Cl. A61B 90/00; A61B 34/20; A61F 2/46; A61B 17/15

(52) U.S. Cl. CPC A61B 90/39; A61B 34/20; A61F 2/4609; A61B 2034/2072; A61B 2034/2051; A61B 2090/3954; A61F 2002/4668; A61B 17/15

(57) ABSTRACT

Magnetically-sensitive surgical tags are attached to an anatomical feature of a subject prior to a surgical procedure. The magnetically-sensitive surgical tags measure the magnitude of a plurality of applied magnetic fields that have respective magnetic field gradients. The magnitudes of the applied magnetic fields are used to determine a first and a second relative three-dimensional position of each magnetically-sensitive surgical tag at first and second times, respectively. When the first and second relative three-dimensional positions of a magnetically-sensitive surgical tag are not substantially the same or within a desired offset distance range, the magnetically-sensitive surgical tag can be repositioned and the new position can be determined and compared to the first position.

### CROSS-REFERENCE TO RELATED APPLICATIONS

This application claims priority to U.S. Provisional Application No. 63/075,980, titled “Precision Surgery Using Smart Surgical Tags,” filed on Sep. 9, 2020, which is hereby incorporated by reference.

### TECHNICAL FIELD

The present application relates generally to medical devices related to surgery.

### BACKGROUND

Orthopedic surgeries that involve the replacement or repair of bones and joints generally require precision to ensure proper alignment with other anatomical features. For example, in total hip replacement, the hip joint is replaced with a prosthetic implant to relieve arthritis pain or to repair a hip fracture. The prosthetic implant needs to be aligned with other anatomical features to ensure that the hip and leg function properly. The center of the hip should normally be aligned with the center of the knee and the center of the ankle. Otherwise, the patient may have bowlegs or knock knees. Examples of normal hip alignment, bowlegs, and knock knees are illustrated in FIG. 1.

Current technologies used to align bone and joints during orthopedic surgical procedures are expensive, inconvenient (e.g., require additional steps to be taken before/during the surgical procedure), and/or inaccurate. In some clinical studies, up to 20-25% of total hip and knee replacement surgeries result in misalignment. Patients with misaligned hips or knees may need to be treated for chronic pain, limp, back pain, sciatica, dislocation, or may need a second surgical procedure (e.g., a revision).

It would be desirable to overcome these and/or other deficiencies in the art.

### SUMMARY

Example embodiments described herein have innovative features, no single one of which is indispensable or solely responsible for their desirable attributes. The following description and drawings set forth certain illustrative implementations of the disclosure in detail, which are indicative of several exemplary ways in which the various principles of the disclosure may be carried out. The illustrative examples, however, are not exhaustive of the many possible embodiments of the disclosure. Without limiting the scope of the claims, some of the advantageous features will now be summarized. Other objects, advantages and novel features of the disclosure will be set forth in the following detailed description of the disclosure when considered in conjunction with the drawings, which are intended to illustrate, not limit, the invention.

An aspect of the invention is directed to a method comprising: securing a plurality of magnetically-sensitive surgical tags to an anatomical feature of a subject, each magnetically-sensitive surgical tag including: a respective magnetic sensor; and a respective data transmitter in electrical communication with the respective magnetic sensor. The method further comprises: producing, with one or more magnetic field gradient coil(s), a magnetic field having a magnetic field gradient with respect to an axis; measuring, with the respective magnetic sensor of each magnetically-sensitive surgical tag at a first time, a respective initial measured magnitude of the magnetic field at a respective first position of each magnetically-sensitive surgical tag; transmitting, with the respective data transmitter, the respective initial measured magnitude from each magnetically-sensitive surgical tag to a computer; measuring, with the respective magnetic sensor of each magnetically-sensitive surgical tag at a second time, a respective subsequent measured magnitude of the magnetic field at a respective second position of each magnetically-sensitive surgical tag; transmitting, with the respective data transmitter, the respective subsequent measured magnitude from each magnetically-sensitive surgical tag to the computer; and determining, with the computer and using the respective initial subsequent measured magnitudes, a respective relative first position and a respective relative second position of each magnetically-sensitive surgical tag along the axis, the respective relative first position and the respective relative second position measured with respect to the one or more magnetic field gradient coil(s).

In one or more embodiments, the method further comprises: comparing, in the computer, the respective relative first position and the respective relative second position of each magnetically-sensitive surgical tag; when a difference between the respective relative first position and the respective relative second position is within a threshold distance, indicating, with the computer, that the respective relative first position and the respective relative second position are the same; and when the difference between the respective relative first position and the respective relative second position is greater than the threshold distance, indicating, with the computer, that the respective relative first position and the respective relative second position are different. In one or more embodiments, the method further comprises: comparing, in the computer, the respective relative first position and the respective relative second position of each magnetically-sensitive surgical tag; when a difference between the respective relative first position and the respective relative second position is within a desired offset distance range, indicating, with the computer, that the respective relative first position and the respective relative second position are the same; and when the difference between the respective relative first position and the respective relative second position is outside of the desired offset distance range, indicating, with the computer, that the respective relative first position and the respective relative second position are different.

In one or more embodiments, the one or more magnetic field gradient coil(s) includes a first magnetic field gradient coil that produces a first magnetic field with respect to the first axis, the respective initial measured magnitude is a first initial measured magnitude, the respective subsequent measured magnitude is a first subsequent measured magnitude, and the method further comprises: producing, with at least a second magnetic field gradient coil, a second magnetic field having a second magnetic field gradient with respect to a second axis that is orthogonal to the first axis; measuring, with the respective magnetic sensor of each magnetically-sensitive surgical tag at a third time, a respective second initial measured magnitude of the second magnetic field at the respective first position of each magnetically-sensitive surgical tag; transmitting, with the respective data transmitter, the respective second initial measured magnitude from each magnetically-sensitive surgical tag to the computer; measuring, with the respective magnetic sensor of each magnetically-sensitive surgical tag at a fourth time, a respective second subsequent measured magnitude of the second magnetic field at the respective second position of each magnetically-sensitive surgical tag; and transmitting, with the respective data transmitter, the respective second subsequent measured magnitude from each magnetically-sensitive surgical tag to the computer. In one or more embodiments, the method further comprises: producing, with at least a third magnetic field gradient coil, a third magnetic field having a third magnetic field gradient with respect to a third axis that is orthogonal to the first and second axes; measuring, with the respective magnetic sensor of each magnetically-sensitive surgical tag at a fifth time, a respective third initial measured magnitude of the third magnetic field at the respective first position of each magnetically-sensitive surgical tag; transmitting, with the respective data transmitter, the respective third initial measured magnitude from each magnetically-sensitive surgical tag to the computer; measuring, with the respective magnetic sensor of each magnetically-sensitive surgical tag at a sixth time, a respective third subsequent measured magnitude of the third magnetic field at the respective second position of each magnetically-sensitive surgical tag; and transmitting, with the respective data transmitter, the respective third subsequent measured magnitude from each magnetically-sensitive surgical tag to the computer.

In one or more embodiments, the method further comprises displaying the respective relative first, second, and third pre-surgical positions and the respective relative first, second, and third post-surgical positions of each magnetically-sensitive surgical tag on a display operatively coupled to the computer. In one or more embodiments, the method further comprises: comparing, in the computer, a respective first three-dimensional relative position of each magnetically-sensitive surgical tag with a respective second three-dimensional relative position of each magnetically-sensitive surgical tag, wherein: the respective first three-dimensional relative position comprises the respective relative first, second, and third initial positions of each magnetically-sensitive surgical tag, and the respective second three-dimensional relative position comprises the respective relative first, second, and third subsequent positions of each magnetically-sensitive surgical tag. When a difference between the respective first three-dimensional relative position and the respective second three-dimensional relative position is less than or equal to a threshold distance with respect to the first, second, or third axis, indicating, with the computer, that the respective first three-dimensional relative position and the respective second three-dimensional relative position are the same. When the difference between the respective first three-dimensional relative position and the respective second three-dimensional relative position is greater than the threshold distance with respect to the first, second, or third axis, indicating, with the computer, that the respective first three-dimensional relative position and the respective second three-dimensional relative position are different.

In one or more embodiments, the method further comprises: comparing, in the computer, a respective first three-dimensional relative position of each magnetically-sensitive surgical tag with a respective second three-dimensional relative position of each magnetically-sensitive surgical tag, wherein: the respective first three-dimensional relative position comprises the respective relative first, second, and third initial positions of each magnetically-sensitive surgical tag, and the respective second three-dimensional relative position comprises the respective relative first, second, and third subsequent positions of each magnetically-sensitive surgical tag. When a straight-line distance between the respective first three-dimensional relative position and the respective second three-dimensional relative position is less than or equal to a threshold distance, indicating, with the computer, that the respective first three-dimensional relative position and the respective second three-dimensional relative position are the same. When the straight-line distance between the respective first three-dimensional relative position and the respective second three-dimensional relative position is greater than the threshold distance, indicating, with the computer, that the respective first three-dimensional relative position and the respective second three-dimensional relative position are different.

In one or more embodiments, the method further comprises: adjusting the respective second three-dimensional relative position of at least one of the magnetically-sensitive surgical tags; after adjusting the respective second three-dimensional relative position of at least one of the magnetically-sensitive surgical tags, sequentially producing the first, second, and third magnetic fields, the first, second, and third magnetic fields having the first, second, and third magnetic field gradients, respectively; measuring, with the respective magnetic sensor of each magnetically-sensitive surgical tag, first, second, and third post-adjustment measured magnitudes of the first, second, and third magnetic fields, respectively, at a respective post-adjustment position of each magnetically-sensitive surgical tag; transmitting, with the respective data transmitter, the first, second, and third post-adjustment measured magnitudes from each magnetically-sensitive surgical tag to the computer; and determining, in the computer, a post-adjustment three-dimensional relative position of each magnetically-sensitive surgical tag based on the first, second, and third post-adjustment measured magnitudes.

In one or more embodiments, the straight-line distance is an initial straight-line distance, and the method further comprises: when a subsequent straight-line distance between the respective first three-dimensional relative position and the post-adjustment three-dimensional relative position is less than or equal to the threshold distance, indicating, with the computer, that the respective first three-dimensional relative position and the post-adjustment three-dimensional relative position are the same; and when the straight-line distance between the respective first three-dimensional relative position and the post-adjustment three-dimensional relative position is greater than the threshold distance, indicating, with the computer, that the respective first three-dimensional relative position and the post-adjustment three-dimensional relative position are different.

In one or more embodiments, the method further comprises performing one or more surgical steps between the first time and the second time. In one or more embodiments, the one or more surgical steps comprises at least a portion of a total hip replacement surgery. In one or more embodiments, the method further comprises, with each magnetically-sensitive surgical tag, wirelessly transmitting the respective initial measured magnitude and the respective subsequent measured magnitude from the respective magnetic sensor to the respective data transmitter.

In one or more embodiments, the data transmitter comprises: an LC circuit; and a power and data management unit (PDMU) electrically coupled to the LC circuit and to the magnetic sensor, the PDMU configured to modulate an impedance of the LC circuit to transmit data to a radio-frequency (RF) coil that is operatively coupled to the computer, and the method further comprises: generating an RF electromagnetic field with the RF coil; and modulating the impedance of the LC circuit to transmit the respective initial measured magnitude and the respective subsequent measured magnitude. In one or more embodiments, the method further comprises wirelessly delivering power to each magnetically-sensitive surgical tag with the RF electromagnetic field.

In one or more embodiments, at least one of the magnetically-sensitive surgical tags is attached to a surgical screw, and the step of securing the at least one of the magnetically-sensitive surgical tags to an anatomical feature of the subject comprises driving the surgical screw into a bone of the subject.

Another aspect of the invention is directed to a method for testing an anatomical fit of an acetabular cup implant, comprising: placing the acetabular cup implant into an acetabulum of a subject, the acetabular cup implant including: a plurality of magnetically-sensitive surgical tags to an anatomical feature of a subject, each magnetically-sensitive surgical tag including: a respective magnetic sensor; and a respective data transmitter in electrical communication with the respective magnetic sensor. The method further comprises: after placing the acetabular cup implant into the acetabulum, sequentially producing first, second, and third magnetic fields, the first, second, and third magnetic fields having first, second, and third magnetic field gradients, respectively, with respect to first, second, and third axes, respectively, that are mutually orthogonal; measuring, with the respective magnetic sensor of each magnetically-sensitive surgical tag, first, second, and third measured magnitudes of the first, second, and third magnetic fields, respectively, at a respective position of each magnetically-sensitive surgical tag; transmitting, with the respective data transmitter, the first, second, and third measured magnitudes from each magnetically-sensitive surgical tag to a computer; determining, in the computer, a respective three-dimensional relative position of each magnetically-sensitive surgical tag based on the first, second, and third measured magnitudes, the respective three-dimensional relative position determined with respect to the first, second, and third magnetic field gradient coils; and determining, in the computer, a measured theta angle and a measured femoral antevision angle of the acetabulum using the respective three-dimensional relative position of each magnetically-sensitive surgical tag.

In one or more embodiments, the method further comprises: comparing, in the computer, the measured theta angle and the measured femoral antevision angle with an ideal theta angle and an ideal femoral antevision angle, respectively; and generating an output, with the computer, that indicates whether the measured theta angle and/or the measured femoral antevision angle is/are substantially the same as the ideal theta angle and/or the ideal femoral antevision angle, respectively.

Yet another aspect of the invention is directed to a medical device comprising: a shaft; a magnetically-sensitive surgical tag disposed at a distal end of the shaft, the magnetically-sensitive surgical tag including: a magnetic sensor; and a data transmitter in electrical communication with the magnetic sensor; a gradient-coil pad comprising a plurality of magnetic field gradient coils; and a computer in electrical communication with the magnetically-sensitive surgical tag and with the gradient-coil pad, wherein: the gradient-coil pad is configured to produce a first magnetic field having a first magnetic field gradient with respect to a first axis, a second magnetic field having a second magnetic field gradient with respect to a second axis, and a third magnetic field having a third magnetic field gradient with respect to a third axis, wherein the first, second, and third axes are mutually orthogonal, the magnetic sensor is configured to measure a magnitude of the first magnetic field, a magnitude of the second magnetic field, and a magnitude of the third magnetic field, and the computer is configured to: determine a relative three-dimensional position of the magnetically-sensitive surgical tag using the magnitude of the first magnetic field, the magnitude of the second magnetic field, and the magnitude of the third magnetic field, the relative three-dimensional position along each axis determined with respect to the gradient-coil pad, and generate an output indicating the relative three-dimensional position of the magnetically-sensitive surgical tag.

In one or more embodiments, the first, second, and third magnetic fields are sequentially produced so as to encode the first, second, and third magnetic field gradients, respectively.

Another aspect of the invention is directed to a medical device comprising: a first body defining a plurality of screw holes, each screw hole configured to receive a surgical screw to attach the first body to a bone of a subject; a second body defining an elongated cutting slot that extends parallel to a cutting axis; a plurality of screws that adjustably attach the first and second bodies to set the cutting axis; a first magnetically-sensitive surgical tag attached to the first body; and a second magnetically-sensitive surgical tag attached to the second body, wherein each of the first and second magnetically-sensitive surgical tags comprises: a respective magnetic sensor; and a respective data transmitter in electrical communication with the respective magnetic sensor.

In one or more embodiments, the medical device further comprises: a gradient-coil pad comprising a plurality of magnetic field coils; and a computer in electrical communication with the first and second magnetically-sensitive surgical tags and with the gradient-coil pad, wherein: the gradient-coil pad is configured to produce a first magnetic field having a first magnetic field gradient with respect to a first axis, a second magnetic field having a second magnetic field gradient with respect to a second axis, and a third magnetic field having a third magnetic field gradient with respect to a third axis, wherein the first, second, and third axes are mutually orthogonal, the respective magnetic sensor is configured to measure a magnitude of the first magnetic field, a magnitude of the second magnetic field, and a magnitude of the third magnetic field at a respective position of a respective magnetically-sensitive surgical tag, and the computer is configured to: determine a relative three-dimensional position of the respective magnetically-sensitive surgical tag using the magnitude of the first magnetic field, the magnitude of the second magnetic field, and the magnitude of the third magnetic field, the relative three-dimensional position along each axis determined with respect to the gradient-coil pad, and generate an output indicating the relative three-dimensional position of the respective magnetically-sensitive surgical tag.

In one or more embodiments, the computer is further configured to: repeatedly determine the relative three-dimensional position of the first magnetically-sensitive surgical tag as the bone is moved to form a virtual sphere, and determine a mechanical axis of the bone based on the virtual sphere. In one or more embodiments, the computer is further configured to determine an angle between the cutting axis and the mechanical axis using the relative three-dimensional position and a relative angle of the second magnetically-sensitive surgical tag.

### BRIEF DESCRIPTION OF THE DRAWINGS

For a fuller understanding of the nature and advantages of the concepts disclosed herein, reference is made to the detailed description of preferred embodiments and the accompanying drawings.

FIG. 1 illustrates an example of normal hip alignment, bowlegs, and knock knees according to the prior art.

FIG. 2 illustrates an embodiment of a system for determining the relative positions and/or orientations (e.g., relative rotational positions) of magnetically-sensitive surgical tags secured to a subject.

FIG. 3 illustrates an example of a magnetic field gradient that can be produced with the system illustrated in FIG. 2.

FIG. 4 is a perspective view of an example gradient-coil stack that can be included in the system illustrated in FIG. 2.

FIG. 5 is a simplified block diagram of one of the magnetically-sensitive surgical tags illustrated in FIG. 2, according to an embodiment.

FIG. 6 is a detailed block diagram of the magnetically-sensitive surgical tag illustrated in FIG. 5.

FIG. 7 is a flow chart of a method for performing a medical procedure, such as surgery, using magnetically-sensitive surgical tags, according to an embodiment.

FIG. 8 is a flow chart of steps 710 and 740 in the flow chart illustrated in FIG. 7, according to an embodiment.

FIGS. 9A-9D illustrate certain steps in the flow chart illustrated in FIG. 7 in the context of total hip replacement surgery.

FIG. 10 is a perspective view of a surgical screw that includes a magnetically-sensitive surgical tag, according to an embodiment.

FIG. 11 is a flow chart of a method for performing an acetabular cup implant surgical procedure using magnetically-sensitive surgical tags according to an embodiment

FIG. 12 illustrates a schematic example of a trial or permanent acetabular cup implant with one or more embedded magnetically-sensitive surgical tag(s) according to an embodiment.

FIG. 13 is a block diagram of a magnetically-sensitive surgical measurement tool according to an embodiment.

FIG. 14 is a perspective view of the magnetically-sensitive surgical measurement tool illustrated in FIG. 13, according to an embodiment.

FIG. 15 illustrates certain points on a pelvis that can be measured with the magnetically-sensitive surgical measurement tool illustrated in FIGS. 13 and 14.

FIG. 16 illustrates example surgical cuts in a total knee replacement surgery.

FIGS. 17A-C illustrates a method for making a distal femoral cut using magnetically-sensitive surgical tags, according to an embodiment.

FIG. 18 is a detailed view of a cutting guide according to an embodiment.

FIG. 19 is a side view of the cutting guide illustrated in FIG. 18 attached to the bottom of the femur.

FIG. 20 is a side view of the cutting guide illustrated in FIG. 18 attached to the bottom of the femur to illustrate a method of determining the mechanical axis of the femur according to an embodiment.

FIGS. 21A, 21 B, and 21C are simplified views of the total magnetic field gradients used for encoding the device location.

FIG. 22 is a schematic top view of the first gradient coil according to an embodiment.

FIG. 23 is a schematic top view of the second gradient coil according to an embodiment.

FIG. 24 is a schematic perspective view of the third gradient coil according to an embodiment.

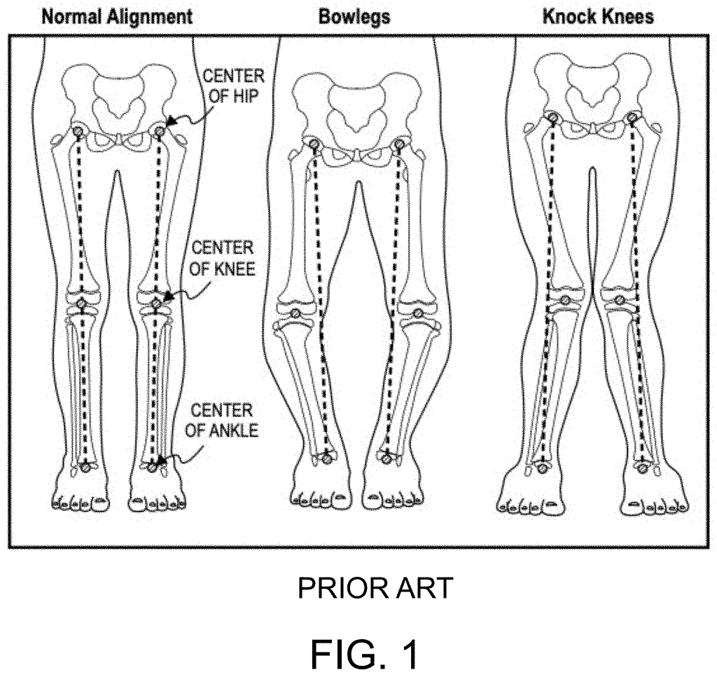

### DETAILED DESCRIPTION

A plurality of magnetically-sensitive surgical tags are attached to an anatomical feature of a subject prior to a surgical procedure. The magnetically-sensitive surgical tags include a magnetic sensor and a data transmitter. The relative three-dimensional position and/or orientation of each magnetically-sensitive surgical tag is determined at a first time, such as prior to the surgical procedure (or one or more surgical steps), and at a second time, such as during or after the surgical procedure (or one or more surgical steps). When the first relative three-dimensional position and/or orientation of a magnetically-sensitive surgical tag is not substantially the same as or within a desired offset range of the second first three-dimensional position and/or orientation of the magnetically-sensitive surgical tag, the position and/or orientation of the magnetically-sensitive surgical tag can be adjusted, such as through a corrective surgical procedure (or one or more corrective surgical steps). The relative three-dimensional position and/or orientation of the magnetically-sensitive surgical tag can be re-checked after repositioning and compared to the first relative three-dimensional position and/or orientation of the magnetically-sensitive surgical tag.

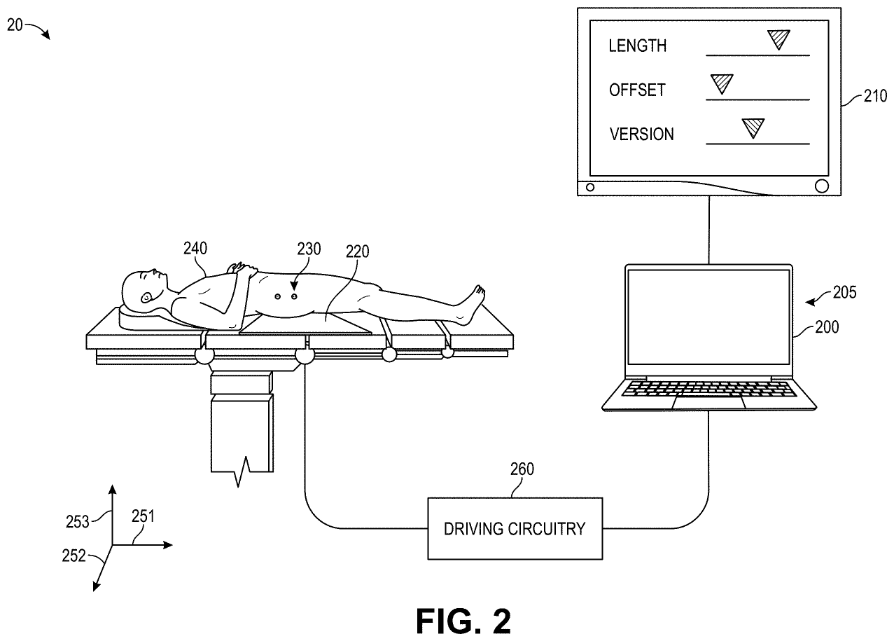

FIG. 2 illustrates an embodiment of a system 20 for determining the relative positions and/or orientations (e.g., relative rotational positions) of magnetically-sensitive surgical tags secured to a subject. The system 20 includes a computer 200, an external display 210, a gradient-coil pad 220, and a plurality of magnetically-sensitive surgical tags 230 secured to a subject 240.

The computer 200 that includes memory that stores software and/or other instructions that cause the computer 200 to control the gradient-coil pad 220 to produce one or more magnetic field gradients. For example, the computer 200 can send control signals that cause the gradient-coil pad 220 to produce at least one magnetic field gradient with respect to a first axis, such as axis 251. The control signals can be sent to the gradient-coil pad 220 and/or to driving circuitry 260 that electrically drives the gradient-coil pad 220. The driving circuitry 260 can include a power source, one or more amplifiers, a controller, and/or other electrical components.

In some embodiments, the controls signals can cause the gradient-coil pad 220 to produce (e.g., sequentially produce) first and second magnetic field gradients with respect to first and second axes, respectively, such as with respect to axes 251 and 252. In a preferred embodiment, the controls signals can cause the gradient-coil pad 220 to produce (e.g., sequentially produce) first, second, and third magnetic field gradients with respect to first, second, and third axes, respectively, such as with respect to axes 251-253. Axes 251-253 are mutually orthogonal or have other predetermined relative orientations. When multiple magnetic field gradients are produced, they can be sequentially produced in a predetermined order that can encode the magnetic field gradients.

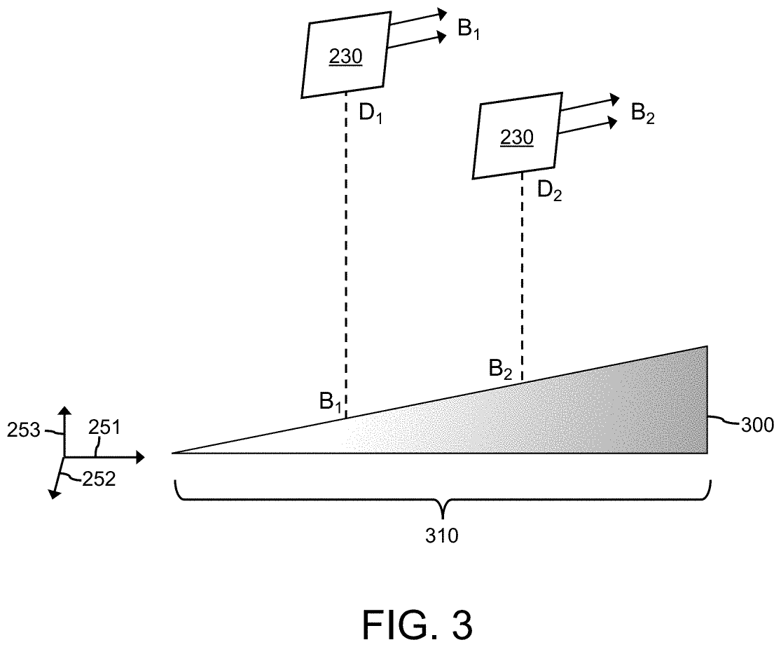

An example of a magnetic field gradient 300 with respect to axis 251 is illustrated in FIG. 3. The gradient-coil pad 220 has an axial field-of-view (FOV) 310, for each magnetic field gradient, along which the magnitude of the magnetic field varies uniquely or monotonically. For example, a first magnetically-sensitive surgical tag 230 at axial position D₁ (e.g., with respect to axis 251) can measure a first unique magnetic field magnitude B₁, and a second magnetically-sensitive surgical tag 230 at axial position D₂ (e.g., with respect to axis 251) can measure a second unique magnetic field magnitude B₂. Thus, the magnetic field magnitude encodes a unique relative axial position within the axial field-of-view of the gradient-coil pad 220 for each magnetic field gradient.

The rotational orientation of a magnetically-sensitive surgical tag 230 can be determined based on the polarity of the measured magnetic field. For example, a positive value of a measured magnetic field can correspond to a first rotational orientation with respect to the axis of the magnetic field gradient (e.g., axis 251 for magnetic field gradient 300). A negative value of a measured magnetic field can correspond to a second rotational orientation with respect to the axis of the magnetic field gradient. A value between the positive and negative values can correspond to a rotational orientation that is between the first and second rotational orientations.

Returning to FIG. 2, the magnetically-sensitive surgical tags 230 are secured and/or attached to a subject using surgical fasteners such as surgical screws, surgical bolts, surgical rivets, surgical staples, and/or surgical tacks. In some embodiments, the magnetically-sensitive surgical tags 230 can be embedded in the surgical fasteners. Each magnetically-sensitive surgical tag 230 includes a magnetic sensor that can measure the magnitude of the magnetic field at a respective position of the magnetically-sensitive surgical tag 230. Each magnetically-sensitive surgical tag 230 sends data representing the measured magnetic fields (e.g., in three dimensions) and/or the magnitude of the magnetic field to the computer 200, which determines the relative axial position of each magnetically-sensitive surgical tag 230 based on the respective magnitude of the magnetic field.

When the gradient-coil pad 220 produces a sequence of magnetic field gradients (e.g., first, second, and third magnetic field gradients), the magnetically-sensitive surgical tags 230 can transmit the data (e.g., representing the measured magnetic fields and/or the magnitude of the magnetic field) corresponding to each magnetic field sequentially (e.g., in real time) or together (e.g., following the last magnetic field gradient in the sequence). When the data is transmitted together, the data can be encoded, such as with a time stamp of the magnetic field measurement, to indicate which magnetic field gradient the data correspond to. In some embodiments, the gradient-coil pad 220 can include at least one RF coil that produces an RF electromagnetic field that can be used to transmit and receive information to/from the magnetically-sensitive surgical tags 230 and/or to wirelessly deliver power to the magnetically-sensitive surgical tags 230.

In other embodiments, the magnetically-sensitive surgical tags 230 can operate at magnetic-field-dependent frequencies (e.g., according to the magnitude of the applied magnetic field sensed by the magnetic sensor) which can be used to determine the relative three-dimensional positions of the magnetically-sensitive surgical tags 230.

In some embodiments, data representing the magnitude of the magnetic fields measured by each magnetically-sensitive tag 230 can be wirelessly sent to an external receiver to determine the three-dimensional positions and/or the relative three-dimensional orientations (e.g., relative rotational positions) of the magnetically-sensitive tags 230. The magnetically-sensitive surgical tag tags 230 can provide up to sub-millimeter positioning and can be powered wirelessly using energy from an applied RF electromagnetic field.

Examples of the magnetically-sensitive surgical tags 230 and associated features are disclosed in U.S. Pat. Nos. 9,915,641 and/or 10,466,227 and/or in U.S. Patent Application Publication Nos. 2021/0141034 and/or 2021/0137412, which are hereby incorporated by reference.

The computer 200 includes software and/or other instructions in its memory to interpret encoded communications signals from the magnetically-sensitive surgical tags 230 that correspond to their identities, relative axial position(s), and/or relative axial orientation(s) (e.g., relative rotational position(s)). The computer 200 can use a mathematical model, a look-up table, or another method to determine the relative axial position of each magnetically-sensitive surgical tag 230. When the magnetically-sensitive surgical tags 230 send data representing the three-dimensional magnetic field measurements, the computer 200 can determine the magnetic field magnitude before determining the relative axial positions of the magnetically-sensitive surgical tags 230. After the relative axial positions are determined, the computer 200 can graphically display the relative axial positions on an internal display 205 or on the external display 210. In addition, the computer 200 can store the relative axial positions and/or the relative rotational position(s) of the magnetically-sensitive surgical tags 230 in memory (e.g., internal or external) that is operatively coupled to the computer 200. In an embodiment, the computer 200 can send data representing the relative axial positions and/or the relative rotational position(s) of the magnetically-sensitive surgical tags 230 to another computer that is operatively coupled to a display screen to display the relative axial positions and/or the relative rotational position(s) of the magnetically-sensitive surgical tags 230.

In some embodiments, the computer 200 can be configured to compare the relative axial positions of the magnetically-sensitive surgical tags 230 with prior or model relative axial positions of the magnetically-sensitive surgical tags 230. For example, the computer can compare pre-surgical and post-surgical relative axial positions of the magnetically-sensitive surgical tags 230 to determine if a prosthetic in a surgical procedure, such as a total hip replacement procedure or a total knee replacement procedure, is placed and/or aligned properly. The computer 200 can determine and display whether any repositioning of the prosthetic is needed with respect to any of the axes.

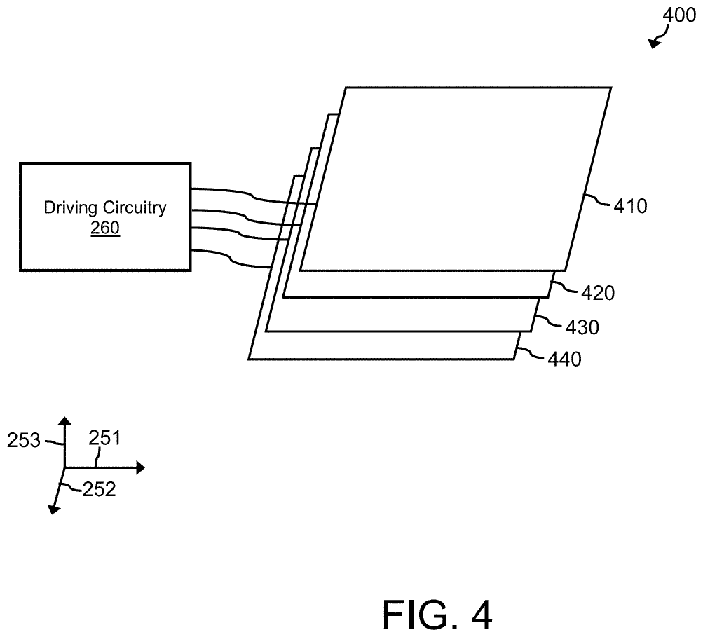

FIG. 4 is a perspective view of an example gradient-coil stack 400 that can be included in the gradient-coil pad 220. The gradient-coil stack 400 includes a first gradient coil 410 that is configured to produce a first magnetic field gradient with respect to the first axis 251, a second gradient coil 420 that is configured to produce a second magnetic field gradient with respect to the second axis 252, and a third gradient coil 430 that is configured to produce a third magnetic field gradient with respect to a third axis 253. Axes 251, 252, 253 can correspond to the Cartesian X , Y, and Z axes, respectively. In some embodiments, the gradient-coil stack 400 is configured to produce the first, second, and/or third magnetic field gradient(s) using a combination of the first, second, and/or third gradient coils 410, 420, 430.

The gradient-coil stack 400 can also include one or more optional RF coil(s) 440 that produce one or more RF electromagnetic field(s). The RF electromagnetic field(s) can be used to send and/or receive signals (e.g., data and/or commands) to/from the magnetically-sensitive surgical tags 230, and/or to wirelessly power the magnetically-sensitive surgical tags 230.

The gradient coils 410, 420, 430 and optional RF coil(s) 440 are electrically coupled to the driving circuitry 260. The driving circuitry 260 can provide power and driving signals to the gradient coils 410, 420, 430 and optional RF coil(s) 440 and can detect data signals sent from the magnetically-sensitive surgical tags 230, which may be in the form of impedance, phase, and/or frequency variations.

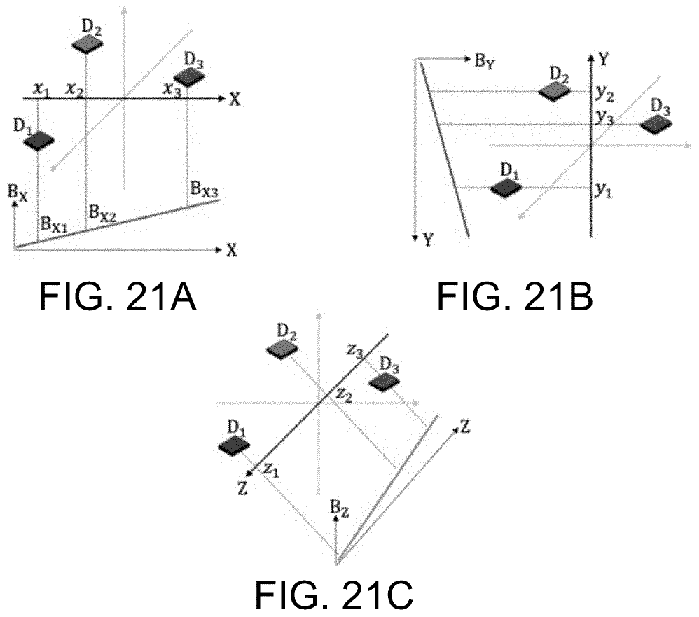

FIGS. 21A-C are simplified views of the total magnetic field gradients used for encoding the device location. Three example magnetic sensor devices D₁, D₂, and D₃ are located in the FOV. To localize the devices D₁, D₂, and D₃ along the X-axis (e.g., the first axis 251), a magnetic field B_{X} having a monotonically-varying magnitude is generated with respect to the X-axis, as illustrated in FIG. 21A. The monotonically-varying magnitude has a gradient in the absolute value of the total magnetic field along the X-axis. The gradient ensures that no two points along the X-axis within the FOV have the same absolute total magnetic field value. For example, the magnitude of the total magnetic field B_{X} measured by devices D₁, D₂, and D₃ can be described according to Equation 1:

∥B_{X1}∥<∥B_{X2}∥<∥B_{X3}∥ (1)

The total magnetic field B_{X} at each device's X location can be described with respect to the X magnetic field contribution from each orthogonal magnetic field at the corresponding X location, as described in Equation 2. The magnetic field gradient with respect to the X axis can be described according to Equation 3.

∥B_{Xi, i=1,2,3}∥=\sqrt{B_{Xi-x}²+B_{Xi-y}²+B_{Xi-z}²} (2

X Gradient=G_{X}=∂B_{X}/∂X (3)

Similarly, to localize the devices D₁, D₂, and D₃ along the Y-axis (e.g., the second axis 252), a magnetic field B_{Y} having a monotonically-varying magnitude is generated with respect to the Y-axis, as illustrated in FIG. 21 B. The monotonically-varying magnitude has a gradient in the absolute value of the total magnetic field along the Y-axis. The gradient ensures that no two points along the Y-axis within the FOV have the same absolute total magnetic field value. For example, the magnitude of the total magnetic field B_{Y} measured by devices D₁, D₂, and D₃ can be described according to Equation 4:

∥B_{Y2}∥<∥B_{Y3}∥<∥B_{Y1}∥ (4)

The total magnetic field B_{Y} at each device's Y location can be described with respect to the Y magnetic field contribution from each orthogonal magnetic field at the corresponding Y location, as described in Equation 5. The magnetic field gradient with respect to the Y axis can be described according to Equation 6.

∥B_{Yi, i=1,2,3}∥=\sqrt{B_{Yi-x}²+B_{Yi-y}²+B_{Yi-z}²} (5)

Y Gradient=G_{Y}=∂B_{Y}/∂Y (6)

Likewise, to localize the devices D₁, D₂, and D₃ along the Z-axis (e.g., the third axis 253), a magnetic field B_{Z} having a monotonically-varying magnitude is generated with respect to the Z-axis, as illustrated in FIG. 21C. The monotonically-varying magnitude has a gradient in the absolute value of the total magnetic field along the Z-axis. The gradient ensures that no two points along the Z-axis within the FOV have the same absolute total magnetic field value. For example, the magnitude of the total magnetic field B_{Z} measured by devices D₁, D₂, and D₃ can be described according to Equation 7:

∥B_{Z1}∥<∥B_{Z2}∥<∥B_{Z3}∥ (7)

The total magnetic field B_{Z} at each device's Z location can be described with respect to the Z magnetic field contribution from each orthogonal magnetic field at the corresponding Z location, as described in Equation 8. The magnetic field gradient with respect to the Y axis can be described according to Equation 9.

∥B_{Zi, i=1,2,3}∥=\sqrt{B_{Zi-x}²+B_{Zi-y}²+B_{Zi-z}²} (8)

Z Gradient=G_{Z}=∂B_{Z}/∂Z (9)

Using these magnetic field measurements along three orthogonal axes, the complete 3D position of each device D₁, D₂, and D₃ can be decoded unambiguously. Since the gradient manifests in the total and absolute magnetic field values along any axis, this localization technique is immune to potential inaccuracies caused by device mis-alignment and orientation mis-match relative to any specific coordinate. As the device orientation changes, the individual field components in Equations 2, 5, and 8 may change but the overall magnitude remains the same.

In order to generate the required spatial gradients in the magnetic field along the three axes, electromagnetic coils (e.g., gradient coils 410, 420, and/or 430) can be designed with one or more of the following design goals: (i) high gradient strength G to achieve high resolution; (ii) planar or substantially planar coils that can be placed close to the patient, such as beneath or in the patient's bed; (iii) enhanced FOV to allow sufficient room for medical procedure navigation, observation, and/or alignment; (iv) high current efficiency to make the maximum use of current drawn by the gradient coils; and/or (v) low coil-length to have less inductance (for fast switching) and less resistance (for lower heating). The gradient coil efficiency η is defined as the ratio of the magnetic field gradient (G) produced by the coil to the current drawn (I). The geometrical design of the coils and static magnetic field simulations can be carried out in a magneto-static software such as Radia, available from the European Synchrotron Radiation Facility. The FOV can be 15 cm×15 cm×10 cm (X Y×Z) though other FOVs can be provided.

The spatial localization resolution (Δx) obtained by the system is given by Equation 10:

Δx=ΔB_{eff}/G (10)

where ΔB_{eff} is the effective resolution that the magnetic sensor can achieve while performing a magnetic field measurement. It is dictated by the noise of the sensing and processing units, most dominant being the quantization noise. G is the applied magnetic field gradient, which is determined by the current in electromagnets and their geometrical structure. There are two predominant noise sources that can cause G to vary from the required ideal value: (a) the offset due to variations in supply current, denoted by δGₛ and (b) the interpolation error caused during gradient characterization, denoted by δGᵢ. To get Δx<100 μm with G=30 mT/m, it is required to have ΔB_{eff}<3 μT. To keep G consistently at 30 mT/m, δGₛ+δGᵢ are targeted to be <1%. In other embodiments, a lower resolution can be provided (e.g., Δx<500 μm).

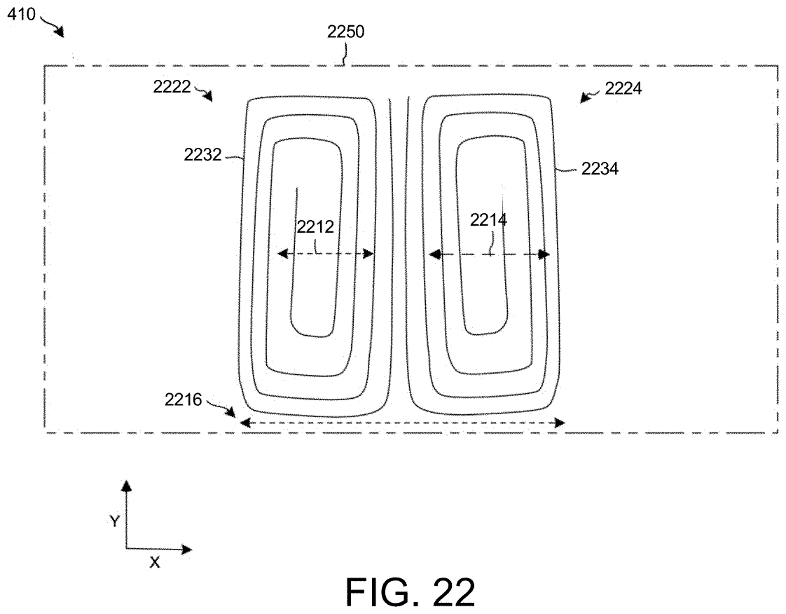

FIG. 22 is a schematic top view of the first gradient coil 410 according to an embodiment. The first gradient coil 410 includes a clockwise spiral winding 2222 and a counterclockwise spiral winding 2224 that are disposed adjacent to or next to each other. The spiral windings 2222, 2224 can be mirror images of each other. Each spiral winding 2222, 2224 has an axis of symmetry 2212, 2214 that is parallel to the first axis 251 (e.g., the X axis). The axes of symmetry 2212, 2214 are aligned in the spiral windings 2222, 2224 to produce a uniform or substantially uniform magnetic field gradient (e.g., a first magnetic field gradient) with respect to the first axis. The spiral windings 2222, 2224 are elongated along the second axis 252 (e.g., the Y axis), such as to form ovals, racetracks (e.g., stadium shapes), rectangles, rounded rectangles, or other elongated shapes. The spiral windings 2222, 2224 can have an elongated length of about 15 cm along the second Y axis which can keep the X-gradient substantially homogenous across the Y FOV. The width 2216 of the first gradient coil 410 is measured along or parallel to the first axis 251 (e.g., the X axis). The length of the first gradient coil 410 is measured along or parallel to the second axis 252 (e.g., the Y axis).

The spiral windings 2222, 2224 are formed by respective wires 2232, 2234 (e.g., first and second wires). Alternatively, more than one wire can be connected together to form a spiral winding. The spiral windings 2222, 2224 have a thickness (e.g., a profile) defined by the thickness of the respective wires 2232, 2234. The wires 2232, 2234 can be identical and thus have the same thickness. Thus, the spiral windings 2222, 2224 have top and bottom planar surfaces (or substantially planar surfaces (e.g., at least 95% planar) that are parallel to X-Y plane 2250. The top and bottom planar surfaces of the spiral windings 2222, 2224 are defined by the respective top and bottom surfaces of wires 2232, 2234. The thickness of the spiral windings 2222, 2224 with respect to the third axis 253 (e.g., the Z axis) is equal to the thickness of the wires 2232, 2234. The wires 2232, 2234 can have an appropriate number of windings or turns to produce the first magnetic field gradient.

The wires 2232, 2234 can be configured to receive a DC current in the range of about 10 A to about 50 A, including about 20 A, about 30 A, and about 40 A, or another current. For example, the wires 2232, 2234 can be copper wires such as Litz 50/32 AWG wires, which denotes 50 strands of 32 AWG wires bundled together. The wires 2232, 2234 have an insulated covering to prevent electrical shorting therebetween.

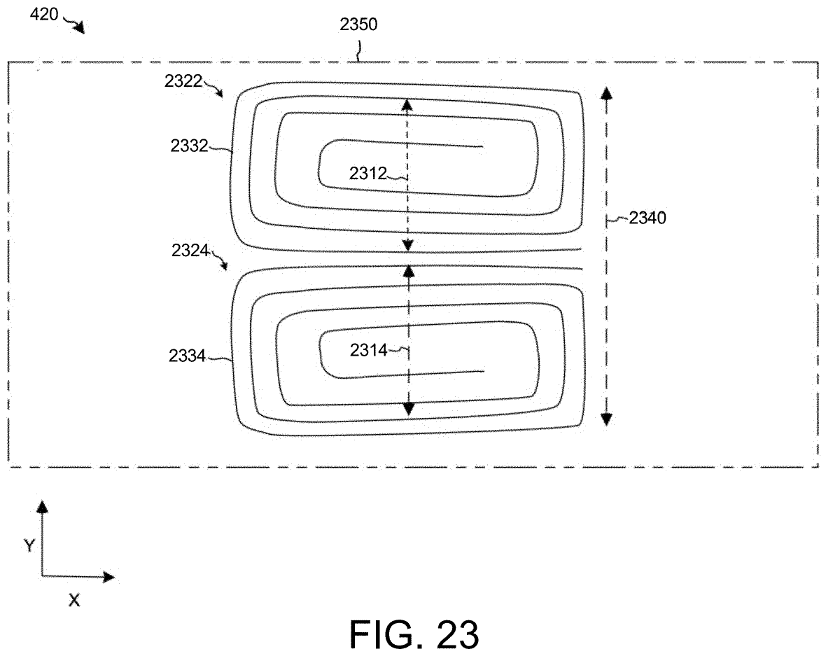

FIG. 23 is a schematic top view of the second gradient coil 420 according to an embodiment. The second electromagnet coil set 420 includes a clockwise spiral winding 2322 and a counterclockwise spiral winding 2324 that are disposed adjacent to or next to each other. The spiral windings 2322, 2324 can be mirror images of each other. Each spiral winding 2322, 2324 has an axis of symmetry 2312, 2314 that is parallel to the second axis 252 (e.g., the Y axis). The axes of symmetry 2312, 2314 are aligned in the spiral windings 2322, 2324 to produce a uniform or substantially uniform magnetic field gradient (e.g., a second magnetic field gradient) with respect to the second axis 252. The second gradient coil 420 is the same as the first gradient coil 410 except that the second gradient coil 420 is rotated by 90 degrees compared to the first gradient coil 410. In other embodiments, the second gradient coil 420 can have other configuration differences compared to the first gradient coil 410.

The spiral windings 2322, 2324 are formed by respective wires 2332, 2334 (e.g., third and fourth wires). Alternatively, more than one wire can be connected together to form a spiral winding. The spiral windings 2322, 2324 have a thickness (e.g., a profile) defined by the thickness of the respective wires 2332, 2334. The wires 2332, 2334 can be identical and thus have the same thickness. Thus, the spiral windings 2322, 2324 have top and bottom planar surfaces (or substantially planar surfaces (e.g., at least 95% planar)) that are parallel to X-Y plane 2350. The top and bottom planar surfaces of the spiral windings 2322, 2324 are defined by the respective top and bottom surfaces of wires 2332, 2334. The thickness of the spiral windings 2322, 2324 with respect to the third axis (e.g., the Z axis) is equal to the thickness of the wires 2332, 2334. The wires 2332, 2334 can have an appropriate number of windings or turns to produce the second magnetic field gradient. The length 2340 of the second gradient coil 420 is measured along or parallel to the second axis 252 (e.g., the Y axis).

The wires 2332, 2334 can be configured to receive a DC current in the range of about 10 A to about 50 A, including about 20 A, about 30 A, and about 40 A, or another current. For example, the wires 2332, 2334 can be Litz 50/32 AWG wires. The wires 2332, 2334 can be the same as or different than the respective wires 2232, 2234.

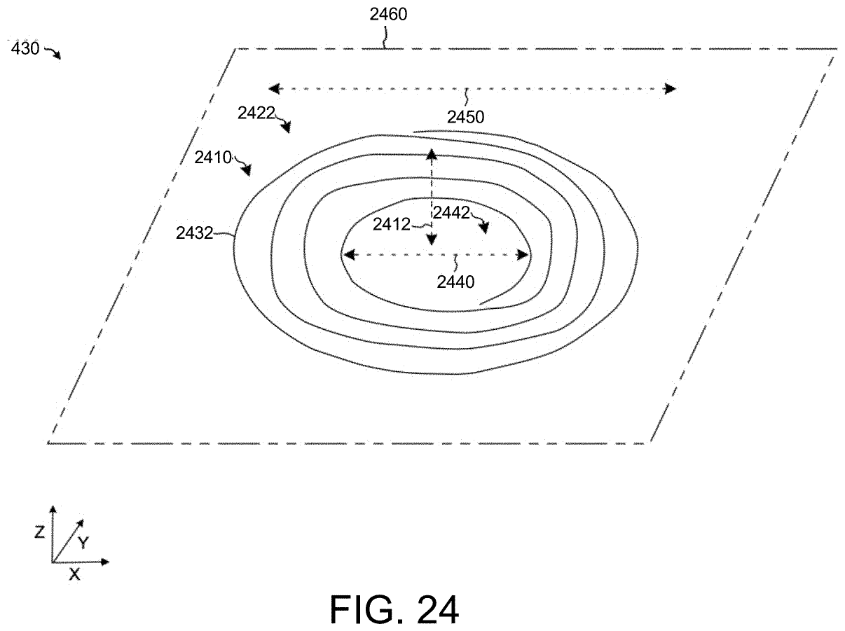

FIG. 24 is a schematic perspective view of the third gradient coil 430 according to an embodiment. The third gradient coil 430 includes a spiral winding 2422 that includes one or more wires 2432 that is/are wound in the shape of an annulus, disc, or ring 2410 (in general, annulus). In an embodiment, two or more wires 2432 are wound next to each other to form the annulus 2410. The wire(s) 2432 are wound in a counter-clockwise direction but in other embodiments the wire(s) 2432 can be wound in a clockwise direction.

The annulus 2410 has an inner diameter 2440 and an outer diameter 2450, where the inner diameter 2440 defines a hollow region or inner cavity 2442 that does not include the wire(s) 2432. The ratio of the outer diameter 2450 to the inner diameter 2440 can be selected to allow an appropriate number of windings or turns of the wire(s) 2432, to produce the third magnetic field gradient. In a specific embodiment, the outer diameter 2450 can be about 28 cm and the inner diameter 2440 can be about 10 cm. The wire(s) 2432 can have an insulated covering to prevent electrical shorting therebetween.

The spiral winding 2422 has an axis of symmetry 2412 that is parallel to the third axis 253 (e.g., the Z axis). The spiral winding 2422 has a thickness (e.g., a profile) defined by the thickness of the wire(s) 2432. Thus, the spiral winding 2422 has top and bottom planar surfaces (or substantially planar surfaces (e.g., at least 95% planar)) that are parallel to X-Y plane 2460. The top and bottom planar surfaces of the spiral winding 2422 are defined by the respective top and bottom surfaces of wire(s) 2432. The thickness of the spiral winding 2422 with respect to the third axis 253 (e.g., the Z axis) is equal to the thickness of the wire(s) 2432. The wire(s) 2432 can have an appropriate number of windings or turns to produce the third magnetic field gradient.

The wire(s) 2432 can be configured to receive a DC current in the range of about 10 A to about 50 A, including about 20 A, about 30 A, and about 40 A, or another current. For example, the wires wire(s) 2432 can be Litz 50/32 AWG wires. The wire(s) 2432 can be the same as or different than wires 2232, 2234, 2332, and/or 2334.

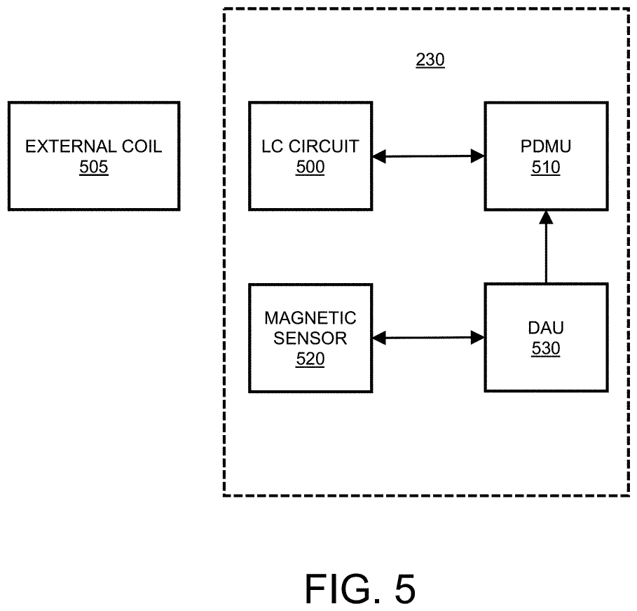

FIG. 5 is a simplified block diagram of magnetically-sensitive surgical tag 230 according to an embodiment. The magnetically-sensitive surgical tag 230 includes an LC circuit 500, a power and data management unit 510, a magnetic sensor 520, and a data acquisition unit 530. The LC circuit 500 is configured to interface with RF electromagnetic fields generated by an external coil 505, which can be same as the RF coil(s) 440 in the gradient-coil stack 400. The power and data management unit 510 can harvest energy from the RF electromagnetic fields (e.g., through wireless power transmission or WPT) to power the magnetically-sensitive surgical tag 230. The magnetic sensor 520 measures the magnetic field at the position of the magnetically-sensitive surgical tag 230 and transmits the sensor data to the data acquisition unit 530. The magnetic sensor 520 can be a 3D magnetic sensor, a 2D magnetic sensor, a 1D magnetic sensor, or another magnetic sensor configured to measure the vector magnitude of a magnetic field. In addition or in the alternative, the magnetic sensor 520 can be a wireless magnetic sensor that wirelessly transmits the sensor data to the data acquisition unit 530.

The data acquisition unit 530 transmits the sensor data from the magnetic sensor 520 to the power and data management unit 510. The power and data management unit 510 modifies an electrical property of the LC circuit 500 to transmit the sensor data to an external system (e.g., gradient-coil pad 220) that includes coil 505. For example, the power and data management unit 510 can modulate the impedance, the phase, and/or the frequency of the LC circuit 500 to transmit the sensor data. In some embodiments, the power and data management unit 510 can include a phased lock loop (PLL) to modify the phase of the LC circuit 500 to transmit the sensor data. Additionally or alternatively, the power and data management unit 510 can include a backscatter modulator that can produce an impedance shift of the LC circuit 500 to transmit the sensor data.

The LC circuit 500, power and data management unit 510, magnetic sensor 520, and data acquisition unit 530 can be on the same substrate (e.g., chip) or two or more substrates.

In one or more embodiments, the data acquisition unit 530 can encode the parameters corresponding to a sensed magnetic field and transmit data indicative of the sensed magnetic field to other components of the invention. That is, the present sensors may achieve the end result described herein by directly transmitting data regarding the sensed magnetic field so that communication of frequency and phase shift information are not required. For example, the magnetically-sensitive surgical tag 230 can include a wireless transceiver, such as a Bluetooth transceiver, a cellular transceiver, or a WiFi transceiver, that can directly transmit data to another device that has its own wireless transceiver, such as a computer (e.g., computer 200), a smartphone, or another device, to receive the data from the magnetically-sensitive surgical tag 230.

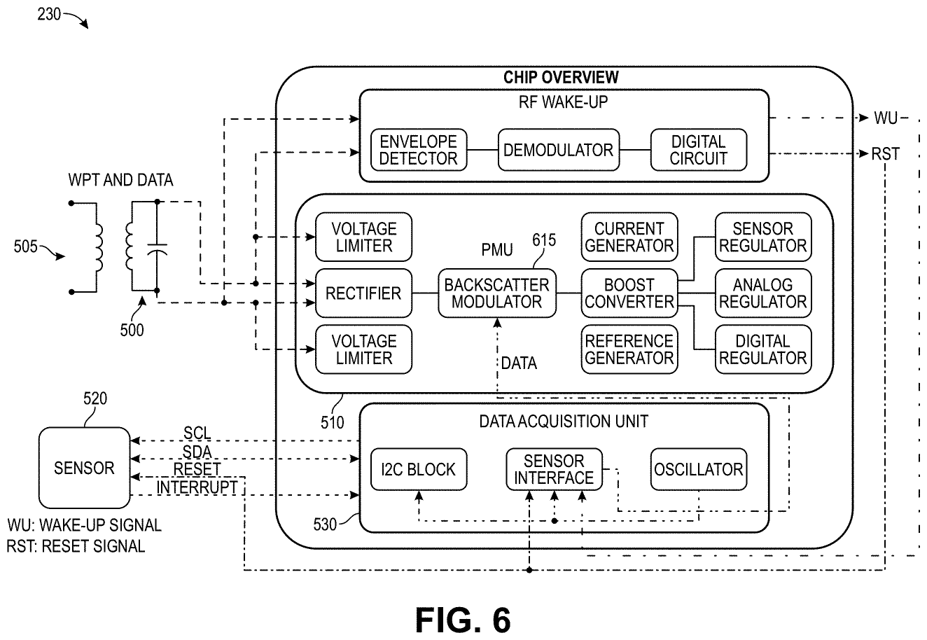

FIG. 6 is a detailed block diagram of the magnetically-sensitive surgical tag 230 according to an embodiment. The power and data management unit 510 in FIG. 6 includes a backscatter modulator 615 that can produce an impedance shift of the LC circuit 500 to transmit the sensor data, as discussed above.

The detailed block diagram illustrated in FIG. 6 includes example details of the magnetically-sensitive surgical tag 230. It is noted that the magnetically-sensitive surgical tag 230 can include additional or fewer components and/or can be configured differently than as illustrated in FIG. 6.

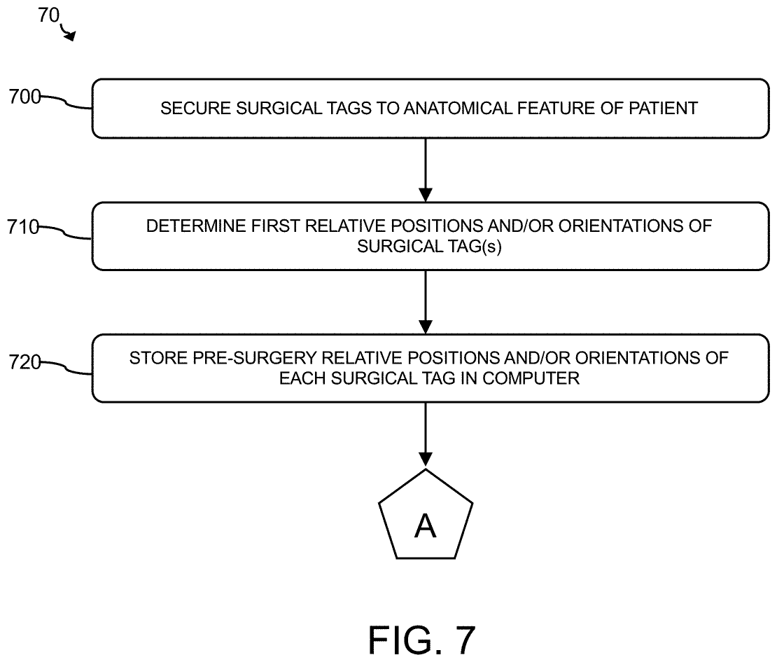

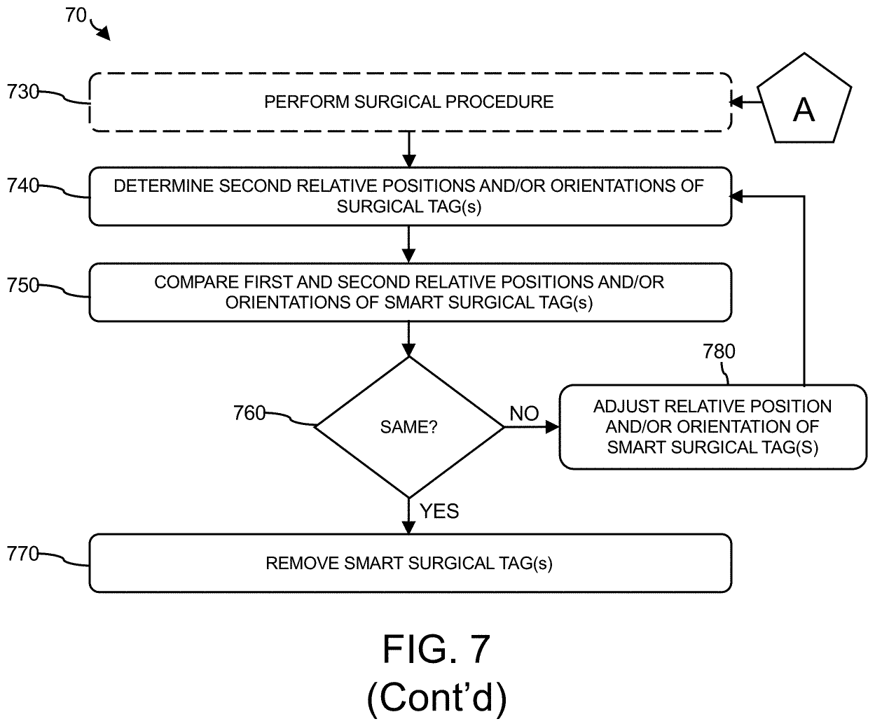

FIG. 7 is a flow chart of a method 70 for performing a medical procedure, such as surgery, using magnetically-sensitive surgical tags 230, according to an embodiment. In step 700, one or a plurality of magnetically-sensitive surgical tags 230 is/are secured to one or more anatomical features of a patient. The magnetically-sensitive surgical tags 230 are preferably secured proximal to a location of a surgical procedure that needs to be monitored, such as for alignment, orientation, or position.

In step 710, a first (e.g., initial) relative position and/or orientation (e.g., relative rotational position) of each magnetically-sensitive surgical tag 230 at a first time is determined, for example using system 20. The first relative position and/or orientation (e.g., relative rotational position) of each magnetically-sensitive surgical tag can be determined with respect to the first axis 251, the second axis 252, and/or the third axis 253, which can correspond to the X, Y, and Z axes, respectively, in the Cartesian coordinate system. In some embodiments, the first time is a time prior to a surgical procedure or prior to one or more surgical steps. The

In step 720, the first relative position and/or orientation (e.g., relative rotational position) of each magnetically-sensitive surgical tag 230 is stored in a computer, such as computer 200. The computer 200 or another computer can optionally display the first relative position and/or orientation (e.g., relative rotational position) of each magnetically-sensitive surgical tag 230 on a display screen operatively coupled to the computer.

In step 730 (via placeholder A), the surgeon optionally performs a surgical procedure or one or more surgical steps. The surgical procedure can be an orthopedic procedure (e.g., a bone or joint replacement) or another surgical procedure. The surgical procedure is performed with the magnetically-sensitive surgical tags in place.

In step 740, a second (e.g., subsequent) relative position and/or orientation (e.g., relative rotational position) of each magnetically-sensitive surgical tag is determined, for example using system 20. The second position and/or orientation is preferably determined with respect to the same number of axes as the first position and/or orientation. The computer 200 or another computer can optionally display the second relative position and/or orientation (e.g., relative rotational position) of each magnetically-sensitive surgical tag 230 on a display screen operatively coupled to the computer.

In step 750, the computer compares the first and second relative positions and/or orientations (e.g., relative rotational positions) of each magnetically-sensitive surgical tag. If the first and second relative positions and/or orientations are the same or within a predetermined tolerance of each other (i.e., step 760=yes), then the magnetically-sensitive surgical tags can be removed in step 770. In some embodiments, the surgeon may desire an offset between the first and second relative positions and/or orientations, in which case the computer determines whether the first and second relative positions and/or orientations are within a desired offset or offset range of each other. The offset or offset range can be with respect to one or more of the axes.

If the first and second positions and/or orientations are the different or outside of a predetermined tolerance or of a desired offset range (i.e., step 760=no), then the surgeon can adjust the position and/or orientation of the magnetically-sensitive surgical tags in step 780 by making surgical adjustments, moving an implant, or performing another surgical technique to indirectly move the magnetically-sensitive surgical tags. In some embodiments, the computer can provide an output that indicates the direction and/or orientation that any of the magnetically-sensitive surgical tags needs to be moved so that the pre- and post-surgery positions and/or orientations are the same or within the desired offset range.

After any magnetically-sensitive surgical tags are re-positioned in step 780, the new (e.g., re-positioned) relative positions and/or relative orientations (e.g., relative rotational positions) of the magnetically-sensitive surgical tags are determined in step 740. The loop of steps 740, 750, 760, and 780 can repeat until the first and new relative positions and/or the first and new relative orientations are the same or within a predetermined tolerance of each other or within a desired offset range (i.e., step 760=yes).

In an alternative embodiment, the surgeon may wish to adjust the position and/or orientation of an anatomical feature that is not in the correct position and/or orientation. For example, a patient may need a surgical procedure to correct a knee knock. In this case, the surgeon would not want the pre- and post-surgery relative positions and/or relative orientations of the magnetically-sensitive surgical tags to be the same. In this embodiment, the surgeon may indicate a desired offset or offset range between the pre-surgery relative positions and/or relative orientations of the magnetically-sensitive surgical tags and the post-surgery relative positions and/or relative orientations of the magnetically-sensitive surgical tags. For example, if the artificial hip needs to be moved in the “Y” direction by 5 mm, the computer can use this offset to determine whether the post-surgery relative position in the “Y” direction is correct (e.g., in step 760).

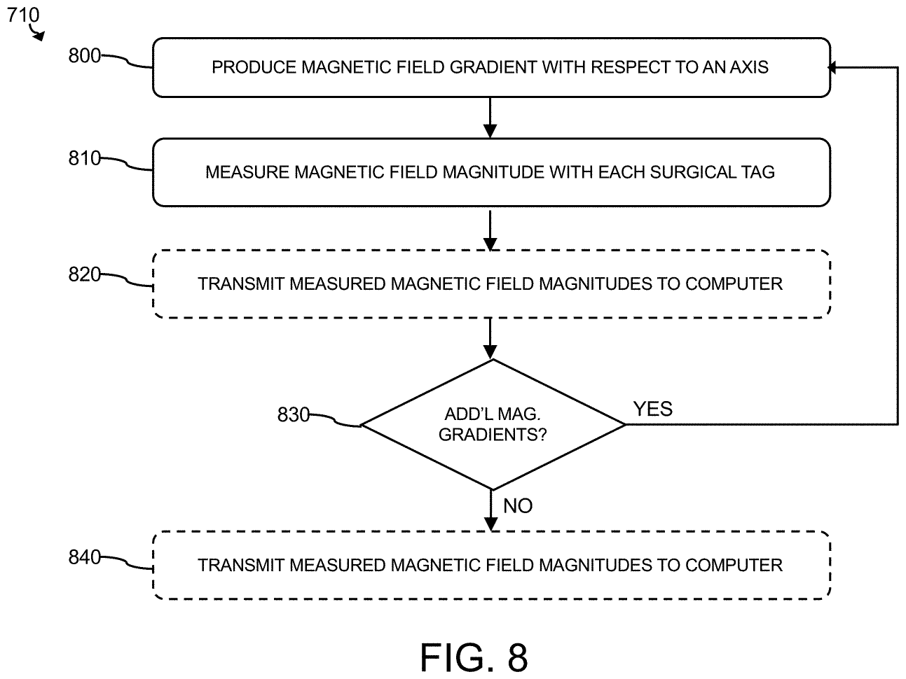

FIG. 8 is a flow chart of steps 710 and 740 (in general step 710) in method 70 according to an embodiment.

In step 800, the gradient-coil pad 220 (e.g., a first gradient coil 410 or multiple gradient coils 410, 420, and/or 430) in system 20 is used to produce a first magnetic field having a first magnetic field gradient with respect to a first axis (e.g., axis 251). The first magnetic field and the first magnetic field gradient can be produced with the gradient-coil pad 220 (e.g., using the first gradient coil 410 or multiple gradient coils 410, 420, and/or 430).

In step 810, the magnetically-sensitive surgical tags 230 measure the magnetic field magnitude at the relative position of each magnetically-sensitive surgical tag 230 with respect to the gradient-coil pad 220. The magnetic field magnitude can be measured directly or determined by measuring the magnetic field with a three-dimensional magnetic field sensor and then calculating the magnetic field magnitude.

In optional step 820, the magnetic field magnitude measured by each magnetically-sensitive surgical tag 230 is optionally transmitted to computer 200 (e.g., via the RF coil(s) 440 in the gradient-coil pad 220).

In step 830, the computer 200 determines whether there are any additional magnetic field gradients to be produced. If so (i.e., step 830=yes), the flow chart returns to step 800 to produce a second magnetic field having a second magnetic field gradient with respect to a second axis (e.g., axis 252). The second magnetic field and the second magnetic field gradient can be produced with the gradient-coil pad 220 (e.g., using the second gradient coil 420 or multiple gradient coils 410, 420, and/or 430). The magnitude of the second magnetic field is measured in step 810 and optionally transmitted to computer 200 in step 820.

This loop can also repeat for the third magnetic field gradient. For example, in step 830, the computer 200 determines whether there are any additional magnetic field gradients to be produced. If so (i.e., step 830=yes), the flow chart returns to step 800 to produce a third magnetic field having a third magnetic field gradient with respect to a third axis (e.g., axis 253). The third magnetic field and the third magnetic field gradient can be produced with the gradient-coil pad 220 (e.g., using the third gradient coil 430 or multiple gradient coils 410, 420, and/or 430). The magnitude of the third magnetic field is measured in step 810 and optionally transmitted to computer 200 in step 820.

When all magnetic field gradients have been produced (i.e., step 830=no), the flow chart proceeds to step 840 where the magnetic field magnitude(s) measured by each magnetically-sensitive surgical tag 230 can be transmitted to computer 200 (e.g., if they were not transmitted in step 820), such as by using the RF coil(s) 440 in the gradient-coil pad 220.

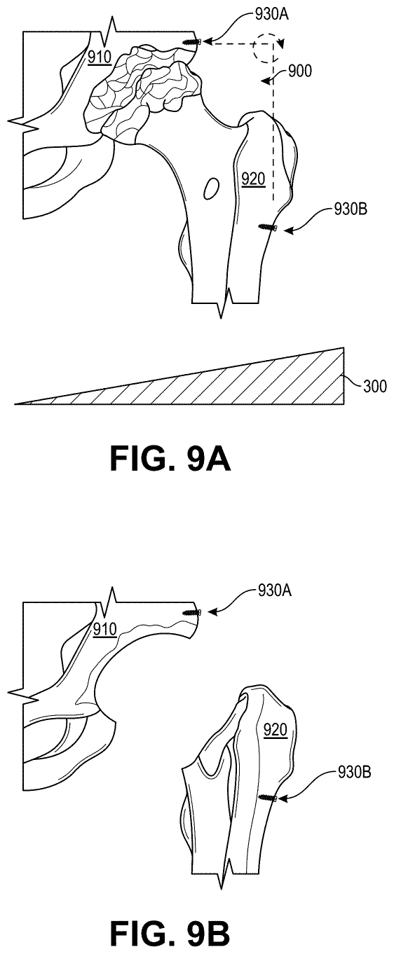

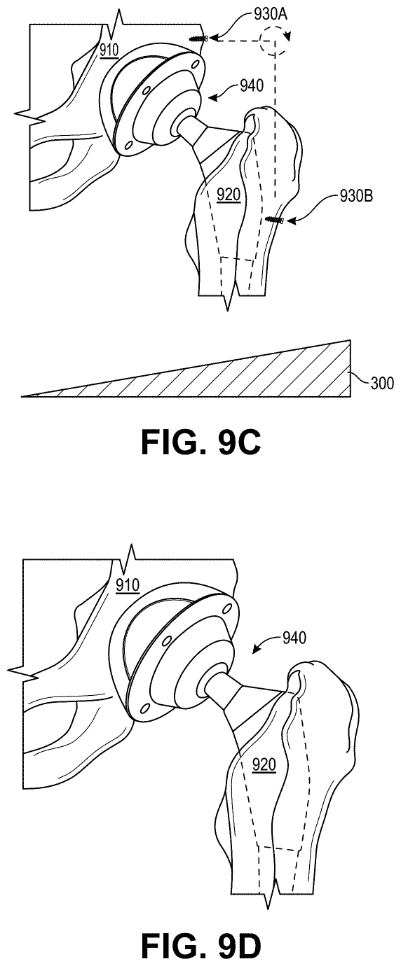

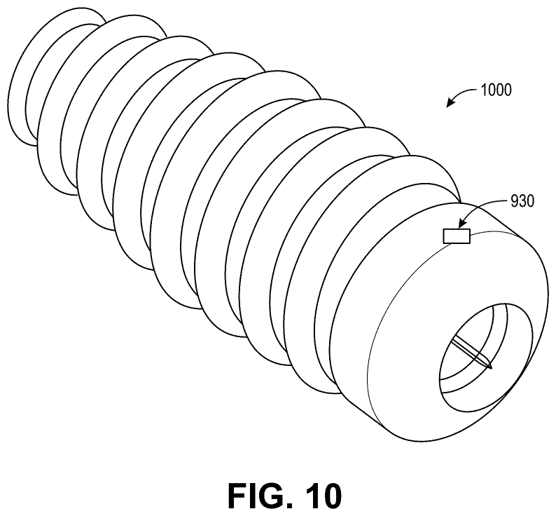

FIG. 9A illustrates an example of steps 700, 710, and 720 in the context of total hip replacement surgery. In this figure, first and second magnetically-sensitive surgical tags 930A, 930B are secured to the pelvis 910 and to the femur bone 920, proximal to the hip joint 900, in advance of a total hip replacement surgery. The magnetically-sensitive surgical tags 930A, 930B can be the same as magnetically-sensitive surgical tags 230. In one example, each magnetically-sensitive surgical tag 930A, 930B (in general, magnetically-sensitive surgical tag 930) is disposed on (e.g., attached to, embedded in, adhered to, etc.) a surgical screw 1000, as illustrated in FIG. 10. The surgical screw 1000 can be screwed (e.g., driven) into the appropriate bone structure (e.g., pelvis 910 and femur bone 920).

In other embodiments, the surgical screw 1000 can be replaced with another mechanical fastener type suitable for a given application such as a bolt, rivet, surgical staple, surgical tack, and so on as would be appreciated by those skilled in the art. Alternatively, the magnetically-sensitive surgical tags 930A, 930B can comprise a housing that is affixed (e.g., using surgical glue, surgical clips, surgical thread, surgical staples, etc.) to an anatomical feature, such as soft tissue (e.g., muscles, tendons, ligaments, fibrous tissue, etc.), in the patient.

After the magnetically-sensitive surgical tags 930A, 930B are secured, the gradient-coil pad 220 in system 20 is used to produce one or more applied magnetic field gradients 300 to determine the relative pre-surgical position(s) and/or orientation(s) (e.g., rotational position(s)) of the magnetically-sensitive surgical tags 930A, 930B with respect to one or more axes (e.g., axes 251, 252, and/or 253), which can be stored in a memory operatively coupled to a computer (e.g., computer 200).

FIGS. 9B and 9C illustrate an example of steps 730 and 740 in the context of total hip replacement surgery. In FIG. 9B, the surgeon has performed conventional surgery to remove the hip joint 900 while the magnetically-sensitive surgical tags 930A, 930B remain secured to the pelvis 910 and to the femur bone 920, respectively. In FIG. 9C, the surgeon has replaced the hip joint 900 with a modular prosthetic implant (trial implant or permanent implant) 940. Proper placement and alignment of the modular prosthetic implant 940 with respect to the pelvis 910 and femur 920 can be determined using the magnetically-sensitive surgical tags 930A, 930B. For example, the modular prosthetic implant 940 can be placed at a first position relative to the pelvis 910 and femur 920. The first position can represent the best approximation of the surgeon based on his/her experience.

Next, the gradient-coil pad 220 in system 20 can be used to produce one or more applied magnetic field gradients 300 to determine the relative post-surgical position(s) and/or orientation(s) (e.g., rotational position(s)) of the magnetically-sensitive surgical tags 930A, 930B with respect to one or more axes (e.g., axes 251, 252, and/or 253), which can be stored in a memory operatively coupled to a computer (e.g., computer 200). The computer (e.g., computer 210 in system 20) can compare the current (e.g., post-surgical) relative three-dimensional positions and/or current relative three-dimensional orientations (e.g., relative rotational positions) of the magnetically-sensitive surgical tags 930A, 930B with the pre-surgery relative three-dimensional positions and/or pre-surgery relative three-dimensional orientations (e.g., relative rotational positions) of the magnetically-sensitive surgical tags 930A, 930B that were determined in step 710 to determine whether the modular prosthetic implant 940 is properly positioned and aligned. If the any of the current relative three-dimensional positions and/or current relative three-dimensional orientations (e.g., relative rotational positions) of the magnetically-sensitive surgical tags 930A, 930B is/are different than the pre-surgery relative three-dimensional positions and/or pre-surgery relative three-dimensional orientations (e.g., relative rotational positions) of the magnetically-sensitive surgical tags 930A, 930B, the computer can generate an output that indicates the direction(s) that one or both of the magnetically-sensitive surgical tags 930A, 930B need to be moved. The surgeon can adjust the position or orientation of the modular prosthetic implant 940 to move the magnetically-sensitive surgical tag(s) 930A, 930B as indicated by the computer output.

In one embodiment, the computer can be programmed to indicate that a post-surgical relative three-dimensional position is different than a pre-surgical relative three-dimensional position of a given magnetically-sensitive surgical tag 930A or 930B only when the difference along at least one axis is greater than a threshold distance. When the difference between the pre- and post-surgical relative three-dimensional positions of the given magnetically-sensitive surgical tag 930A or 930B along each axis is less than or equal to the threshold distance, the computer can be programmed to indicate that the pre- and post-surgical relative three-dimensional positions are the same (or substantially the same).

In another embodiment, the computer can be programmed to indicate that the post-surgical relative three-dimensional position is different than the pre-surgical relative three-dimensional position of a given magnetically-sensitive surgical tag 930A or 930B only when the sum of the differences (e.g., the sum of the absolute value of the differences) between the pre- and post-surgical relative three-dimensional positions along each axis is greater than a threshold difference. When the sum of the differences (e.g., the sum of the absolute value of the differences) between the pre- and post-surgical relative three-dimensional positions along each axis is less than or equal to the threshold difference, the computer can be programmed to indicate that the pre- and post-surgical relative three-dimensional positions are the same (or substantially the same).

In another embodiment, the computer can be programmed to indicate that the post-surgical relative three-dimensional position is different than the pre-surgical relative three-dimensional position of a given magnetically-sensitive surgical tag 930A or 930B only when a straight-line distance between the pre- and post-surgical relative three-dimensional positions is greater than a threshold distance. When the straight-line distance between the pre- and post-surgical relative three-dimensional positions is less than or equal to the threshold distance, the computer can be programmed to indicate that the pre- and post-surgical relative three-dimensional positions are the same (or substantially the same).

After the surgeon adjusts the position or orientation of the modular prosthetic implant 940, such as by performing a corrective surgical procedure, the surgeon can confirm that the new position/orientation is correct by re-checking the current (new) relative three-dimensional positions and/or current relative three-dimensional orientations (e.g., relative rotational positions) of each magnetically-sensitive surgical tag 930A, 930B, and can then compare the current relative three-dimensional positions and/or current relative three-dimensional orientations (e.g., relative rotational positions) of the magnetically-sensitive surgical tags 930A, 930B with the pre-surgery relative three-dimensional positions and/or pre-surgery relative three-dimensional orientations (e.g., relative rotational positions) of the magnetically-sensitive surgical tags 930A, 930B that were determined in step 710. This loop (e.g., equivalent to step 760=no) can continue until the computer determines that the pre- and post-surgery relative three-dimensional positions and/or relative three-dimensional orientations (e.g., relative rotational positions) of the magnetically-sensitive surgical tags 930A, 930B are the same or substantially the same (e.g., with a predetermined tolerance level (e.g., plus or minus 5%) of each other). When the pre- and post-surgery relative three-dimensional positions and/or relative three-dimensional orientations (e.g., relative rotational positions) of the magnetically-sensitive surgical tags 930A, 930B are the same or substantially the same, the surgeon can remove the magnetically-sensitive surgical tags 930A, 930B, as illustrated in FIG. 9D.

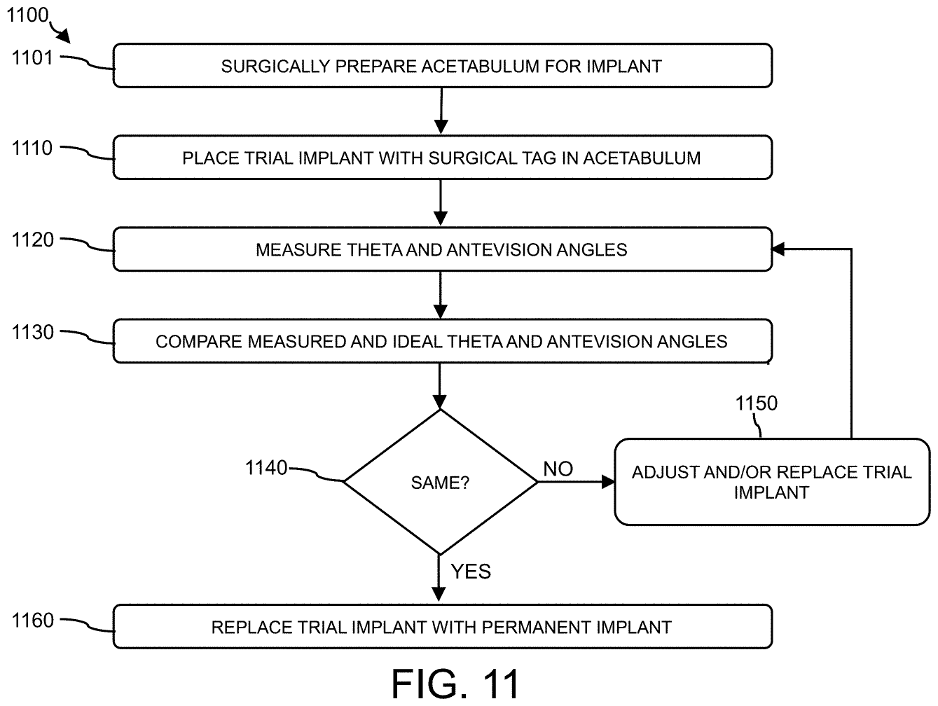

FIG. 11 is a flow chart of a method 1100 for performing an acetabular cup implant surgical procedure using magnetically-sensitive surgical tags according to an embodiment.

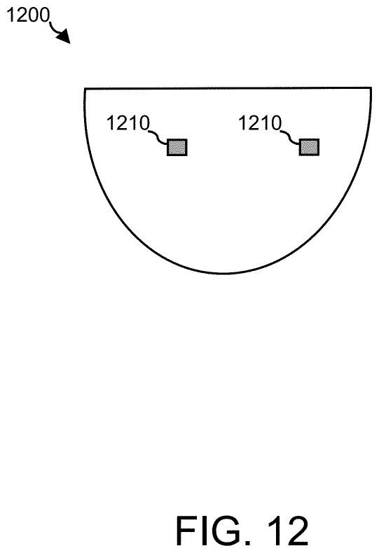

In step 1101, a surgeon reams and prepares the patient's acetabulum for an acetabular cup implant, which can be part of a hip replacement surgery. In step 1110, a trial acetabular cup implant with one or more embedded magnetically-sensitive surgical tag(s) is inserted into the patient's acetabulum. FIG. 12 illustrates a schematic example of a trial or permanent acetabular cup implant 1200 with one or more embedded magnetically-sensitive surgical tag(s) 1210 according to an embodiment. The trial or permanent acetabular cup implant 1200 can include additional or fewer magnetically-sensitive surgical tags 1210. The magnetically-sensitive surgical tags 1210 can be adhered to, attached to, or otherwise physically connected to the trial or permanent acetabular cup implant 1200. Each magnetically-sensitive surgical tag 1210 can be the same as magnetically-sensitive surgical tag 230. The trial or permanent acetabular cup implant 1200 can be used with system 20.

In step 1120, the surgeon measures the theta (inclination) and femoral antevision angles of the acetabulum when the trial acetabular cup implant is inserted into the patient's acetabulum. The theta and antevision angles can be measured using the embedded magnetically-sensitive surgical tag(s) 1210 by applying one or more magnetic fields having respective magnetic field gradients along respective orthogonal axes, for example by using system 20. The magnitude of the measured magnetic field associated with each magnetic field gradient can be used to determine the relative positions and/or orientations (e.g., relative rotational positions) of each magnetically-sensitive surgical tag(s) 1210 with respect to the axis of the magnetic field gradient.

For example, since the magnetically-sensitive surgical tags 1210 provide vector data in 3 dimensions, a plane can be determined to measure theta and anteversion in relation to the gradient-coil pad 220 and/or to the gradient-coil stack 400. Alternatively, the anteversion and theta angles can also be calculated relative to pelvic tilt measured utilizing the magnetically sensitive surgical measurement tool 1300 discussed below.

In step 1130, the measured theta and antevision angles are compared (e.g., using a computer such as computer 200) with the ideal theta and antevision angles, respectively. The ideal theta angle is 40° and the ideal antevision angle is 15° . If the measured theta and antevision angles are not substantially the same as (e.g., greater than 5° of) the ideal theta and antevision angles (i.e., step 1140=no), respectively, then the trial acetabular cup implant is sized and/or placed incorrectly. The computer (e.g., computer 200) can generate an output that indicates whether the measured theta angle and/or the measured antevision angle is substantially the same as the ideal theta angle and the ideal antevision angles, respectively. The surgeon can adjust the position of the trial acetabular cup implant and/or can replace the trial acetabular cup implant with one having a different size and/or shape in step 1150.

After the trial acetabular cup implant has been repositioned and/or replaced, the theta and antevision angles can be re-measured in step 1120 and compared to the ideal theta and antevision angles, respectively. The loop of steps 1120, 1130, 1140, and 1150 can be repeated until the measured theta and antevision angles (e.g., with the trial acetabular cup implant inserted) are substantially the same as the ideal theta and antevision angles, respectively.

When the measured theta and antevision angles are substantially the same as (e.g., within 0° to 5° of) the ideal theta and antevision angles (i.e., step 1140=yes), respectively, then the trial acetabular cup implant is sized and placed correctly and the trial acetabular cup implant can be replaced with a permanent implant in step 1160. The permanent acetabular cup implant can be sized to be identical (or substantially identical) to the trial acetabular cup implant such that the theta and antevision angles will be the same as measured in step 1120.

In some embodiments, the permanent implant can include one or more embedded magnetically-sensitive surgical tag(s). After the permanent implant is inserted, the theta and antevision angles can optionally be re-measured using the embedded magnetically-sensitive surgical tag(s) in the same manner as in the trial acetabular cup implant. Appropriate adjustments can be made to the permanent implant when the measured theta and antevision angles are not substantially the same as (e.g., within 0° to 5° of) the ideal theta and antevision angles, respectively. In an alternative embodiment, only the permanent acetabular cup implant includes magnetically-sensitive surgical tag(s) and the trial acetabular cup implant does not include magnetically-sensitive surgical tag(s). Therefore, the magnetically-sensitive surgical tag(s) may be placed in or on any combination of the trial acetabular cup implant, the permanent implant, and/or surgical tools used in the procedure.

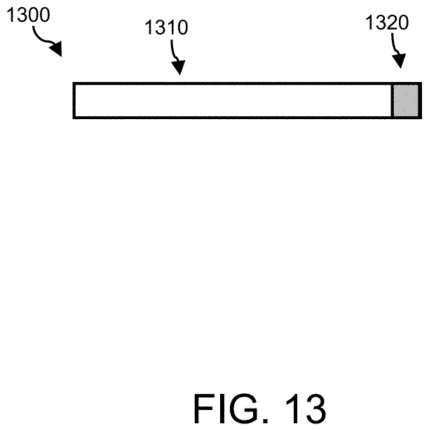

FIG. 13 is a block diagram of a magnetically-sensitive surgical measurement tool 1300 according to an embodiment. The magnetically-sensitive surgical measurement tool 1300 includes a shaft 1310 and a magnetically-sensitive surgical tag 1320 attached to the distal end of the shaft 1310. The shaft 1310 comprises plastic and/or a non-ferromagnetic metal (e.g., aluminum) such that the shaft material does not interfere with the magnetic sensor(s) on the magnetically-sensitive surgical tag 1320. The magnetically-sensitive surgical measurement tool 1300 can be used to determine the relative position of an anatomical feature prior to or during a clinical procedure. For example, a user can hold the proximal end of the magnetically-sensitive surgical measurement tool 1300 and place the distal end on or adjacent to an anatomical feature. The magnetically-sensitive surgical tag 1320 is then used to measure the relative position and/or orientation (e.g., relative rotational position) of the anatomical feature (e.g., by measuring the relative position and/or orientation of the magnetically-sensitive surgical tag 1320 located on or adjacent to the anatomical feature), for example by using system 20.

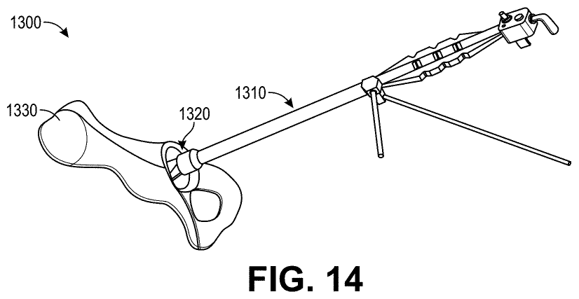

FIG. 14 is a perspective view of the magnetically-sensitive surgical measurement tool 1300 according to an embodiment. In this embodiment, the magnetically-sensitive surgical measurement tool 1300 is used to insert an implant (e.g., an acetabular cup implant) as part of a hip replacement surgery. The distal end of the magnetically-sensitive surgical measurement tool 1300 can be round (e.g., spherical) to engage the implant. In some embodiments, the magnetically-sensitive surgical measurement tool 1300 can be used to measure the theta and antevision angles of the acetabulum 1330 instead of or in addition to the magnetically-sensitive surgical tag(s) 1210 on the trial or permanent implant 1200, as discussed above.

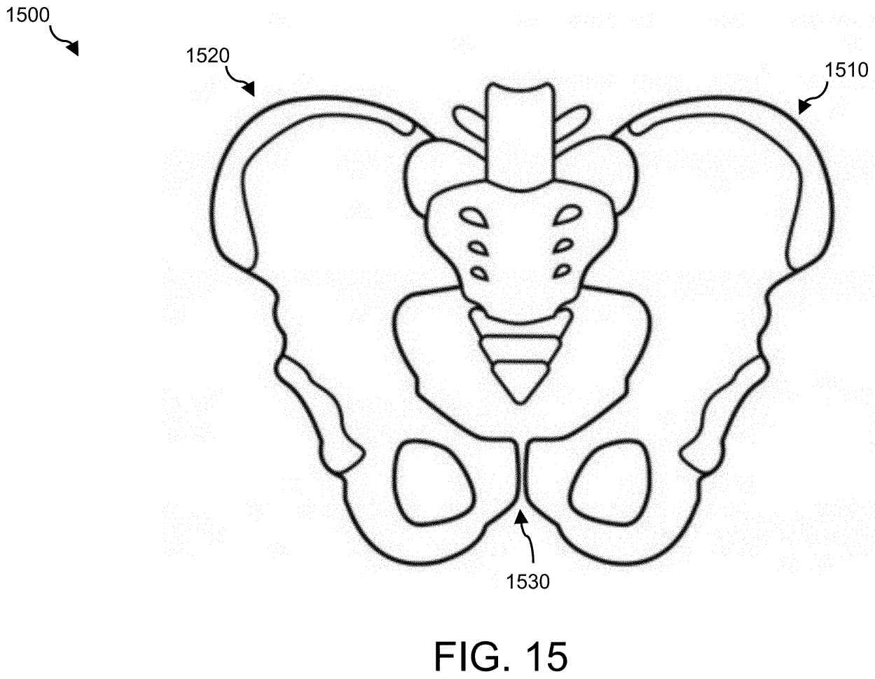

In a specific example, the magnetically-sensitive surgical measurement tool 1300 can be used to measure the relative positions of multiple points on the pelvis, which can be used to define a pelvic plane. In one embodiment, the points on the pelvis 1500 include the left and right iliac crests 1510, 1520, respectively, and the pubic bone 1530, as illustrated in FIG. 15.

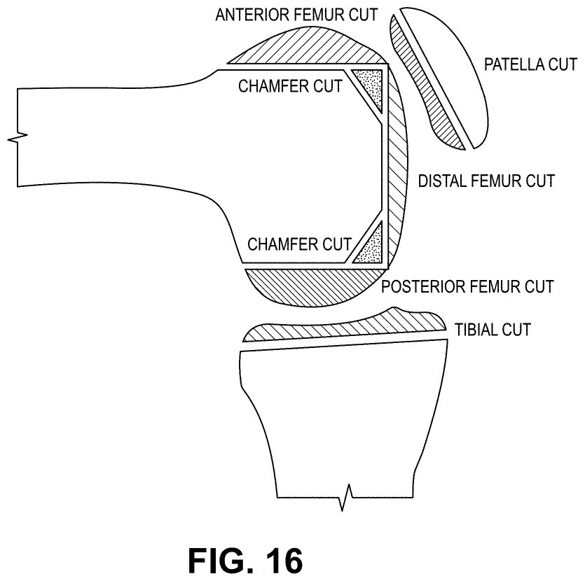

Another application of the magnetically-sensitive surgical tags is to determine the relative anatomical angles for performing surgical cuts or incisions. For example, the magnetically-sensitive surgical tags can be used to determine the angle for any of the cuts in a total knee replacement surgery, such as those illustrated in FIG. 16.

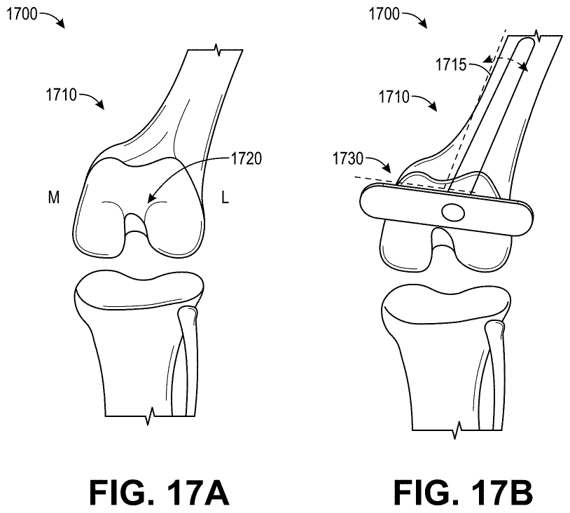

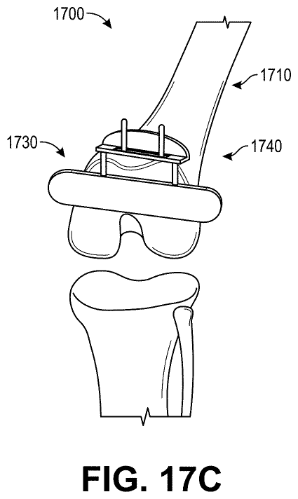

FIGS. 17A-C illustrates a method 1700 for making a distal femoral cut using magnetically-sensitive surgical tags according to an embodiment. In FIG. 17A, the femur 1710 is prepared by drilling a hole down the medullary canal 1720. In FIG. 17B, an intra-medullary guide 1730 is aligned with respect to the anatomical axis 1715 of the femur. In some embodiments, the intra-medullary guide 1730 can be aligned about 6° off of the anatomical axis 1715. In FIG. 17C, a cutting guide 1740 is placed above the intra-medullary guide 1730 to make the distal femoral cut. The cutting guide 1740 includes a slot through which the distal femoral cut is made. The intra-medullary guide 1730 and/or the cutting guide 1740 can include magnetically-sensitive surgical tags to measure the appropriate angles.

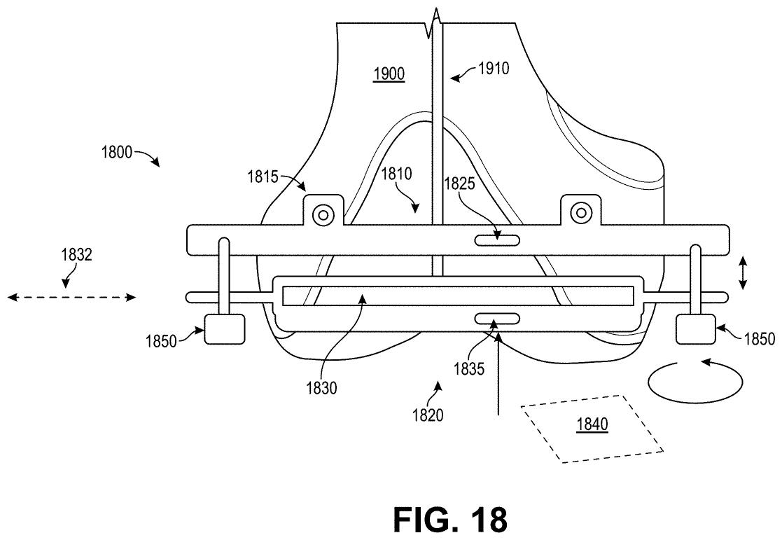

FIG. 18 is a detailed view of a cutting guide 1800 according to an embodiment. The cutting guide 1800 can be the same as cutting guide 1740. The cutting guide 1800 includes a plurality of magnetically-sensitive surgical tags 1825, 1835. The first magnetically-sensitive tag 1825 is attached to a first body 1810 of the cutting guide 1800. The second magnetically-sensitive tag 1835 is attached to a second body 1820 of the cutting guide 1800.

A plurality of screw holes 1815 are defined in the first body 1810 of the cutting guide 1800. Surgical screws can be inserted through the screw holes 1815 to secure the first body 1810 to a bone such as the femur 1900. The relative orientation of the cutting guide 1800 with respect to the mechanical axis 1910 of the femur 1900 can be determined using the first magnetically-sensitive surgical tag 1825. The mechanical axis 1910 can be determined using the magnetically-sensitive surgical tags (e.g., as described below) or manually, for example as described below.

The second body 1820 defines an elongated cutting slot 1830 that extends parallel to a cutting slot axis 1832. The cutting slot 1830 defines a cutting guide plane 1840 along which a surgical cut can be made into the femur 1900. The cutting guide plane 1840 is preferably orthogonal (e.g., 90°) or approximately orthogonal (e.g., 90° plus or minus 3°) with respect to the mechanical axis 1910 when the cutting guide 1800 is secured to the femur 1900. The relative orientation or angle of the cutting guide plane 1840 with respect to the mechanical axis 1910 can be measured using the second magnetically-sensitive tag 1835. When the cutting guide plane 1840 is not approximately orthogonal (e.g., 90° plus or minus 3°) to the mechanical axis 1910, the angle of the cutting slot 1830 can be adjusted using an angle adjustment mechanism. For example, the second body 1820 can be adjustably attached to the first body 1810 using thumb screws 1850 on opposing sides of the cutting slot 1830. The thumb screws 1850 can be tightened or loosened to adjust the relative angle of the cutting slot axis 1832 with respect to the mechanical axis 1910, thereby adjusting the relative position of the cutting guide plane 1840 with respect to the mechanical axis 1910. The thumb screws 1850 can be configured to adjust the relative angle of the cutting slot axis 1832 with respect to two axes: the varus/valgus (bowlegs/knock knees, respectively) plane and the flexion/extension (e.g., sagittal) plane.

After using the angle adjustment mechanism, the angle of the cutting guide plane 1840 with respect to the mechanical axis 1910 can be re-measured using the second magnetically-sensitive surgical tag 1835. This process can repeat as needed until the cutting guide plane 1840 is approximately orthogonal with respect to the mechanical axis 1910. The cutting slot 1830 of the cutting guide 1800 can then be used to make a distal femoral cut.

The relative three-dimensional position and/or orientations (e.g., relative rotational positions) of the first and second magnetically-sensitive tag 1825, 1835 can be measured using system 20.

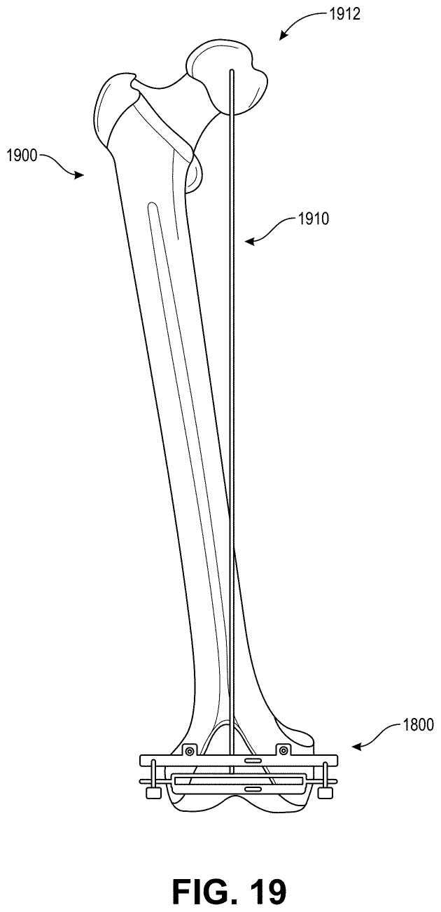

FIG. 19 illustrates the cutting guide 1800 attached to the bottom of the femur 1900. The mechanical axis 1910 is defined between the femoral head 1912 and the center of the distal end of the femur 1900.

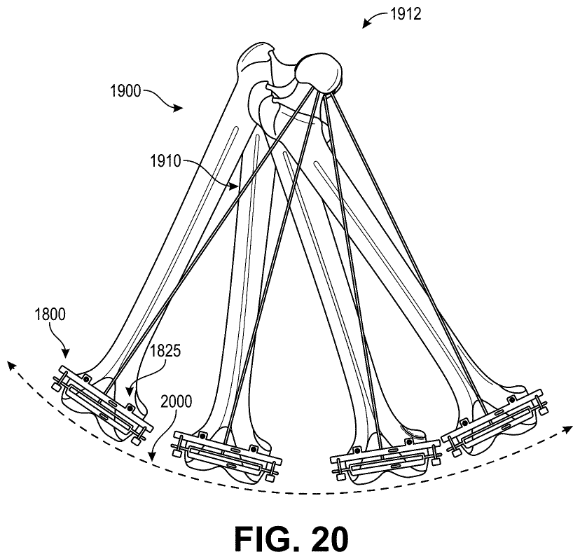

FIG. 20 illustrates that the magnetically-sensitive surgical tag 1825 on the cutting guide 1800 can be used to determine the mechanical axis 1910 of the femur 1900. The cutting guide 1800 is first secured to the femur 1900 using surgical screws. Next, the femur 1900 is pivoted and/or placed through a range of motions while the relative three-dimensional position of the first magnetically-sensitive surgical tag 1825 is measured. The relative three-dimensional position of the first magnetically-sensitive surgical tag 1825 at position defines a virtual sphere 2000 over which the mechanical axis 1910 sweeps as the femur 1900 is pivoted. A computer (e.g., computer 200 in system 20) can be programmed to calculate the virtual sphere 2000 and to determine the mechanical axis 1910 using the plurality of relative three-dimensional positions of the first magnetically-sensitive surgical tag 1825. For example, the mechanical axis 1910 can be defined as the radius of the virtual sphere 2000. The cutting guide plane 1840 can be tangential to the point on the virtual sphere 2000 at the center of the knee (e.g., at femoral head 1912).

After the mechanical axis 1910 is determined, the second magnetically-sensitive surgical tag 1835 can be used to determine the angle between the cutting-guide plane 1840 and the mechanical axis 1910, as discussed above. When the cutting guide plane 1840 is not approximately orthogonal to the mechanical axis 1910, the angle of the cutting slot 1830 can be adjusted using an angle adjustment mechanism such as thumb screws 1850.

The invention should not be considered limited to the particular embodiments described above. Various modifications, equivalent processes, as well as numerous structures to which the invention may be applicable, will be readily apparent to those skilled in the art to which the invention is directed upon review of this disclosure. The above-described embodiments may be implemented in numerous ways. One or more aspects and embodiments involving the performance of processes or methods may utilize program instructions executable by a device (e.g., a computer, a processor, or other device) to perform, or control performance of, the processes or methods.

In this respect, various inventive concepts may be embodied as a non-transitory computer readable storage medium (or multiple non-transitory computer readable storage media) (e.g., a computer memory of any suitable type including transitory or non-transitory digital storage units, circuit configurations in Field Programmable Gate Arrays or other semiconductor devices, or other tangible computer storage medium) encoded with one or more programs that, when executed on one or more computers or other processors, perform methods that implement one or more of the various embodiments described above. When implemented in software (e.g., as an app), the software code may be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smartphone or any other suitable portable or fixed electronic device.

Also, a computer may have one or more communication devices, which may be used to interconnect the computer to one or more other devices and/or systems, such as, for example, one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, a computer may have one or more input devices and/or one or more output devices. These devices can be used, among other things, to present a user interface. Examples of output devices that may be used to provide a user interface include printers or display screens for visual presentation of output and speakers or other sound generating devices for audible presentation of output. Examples of input devices that may be used for a user interface include keyboards, and pointing devices, such as mice, touch pads, and digitizing tablets. As another example, a computer may receive input information through speech recognition or in other audible formats.

The non-transitory computer readable medium or media may be transportable, such that the program or programs stored thereon may be loaded onto one or more different computers or other processors to implement various one or more of the aspects described above. In some embodiments, computer readable media may be non-transitory media.

The terms “program,” “app,” and “software” are used herein in a generic sense to refer to any type of computer code or set of computer-executable instructions that may be employed to program a computer or other processor to implement various aspects as described above. Additionally, it should be appreciated that, according to one aspect, one or more computer programs that when executed perform methods of this application need not reside on a single computer or processor, but may be distributed in a modular fashion among a number of different computers or processors to implement various aspects of this application.

Computer-executable instructions may be in many forms, such as program modules, executed by one or more computers or other devices. Generally, program modules include routines, programs, objects, components, data structures, etc. that perform particular tasks or implement particular abstract data types. The functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Thus, the disclosure and claims include new and novel improvements to existing methods and technologies, which were not previously known nor implemented to achieve the useful results described above. Users of the method and system will reap tangible benefits from the functions now made possible on account of the specific modifications described herein causing the effects in the system and its outputs to its users. It is expected that significantly improved operations can be achieved upon implementation of the claimed invention, using the technical components recited herein.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

What is claimed is:

1. A method comprising:
securing a plurality of magnetically-sensitive surgical tags to an anatomical feature of a subject, each magnetically-sensitive surgical tag including:
a respective magnetic sensor; and
a respective data transmitter in electrical communication with the respective magnetic sensor;
producing, with one or more magnetic field gradient coil(s), a magnetic field having a magnetic field gradient with respect to an axis;
measuring, with the respective magnetic sensor of each magnetically-sensitive surgical tag at a first time, a respective initial measured magnitude of the magnetic field at a respective first position of each magnetically-sensitive surgical tag;
transmitting, with the respective data transmitter, the respective initial measured magnitude from each magnetically-sensitive surgical tag to a computer;
measuring, with the respective magnetic sensor of each magnetically-sensitive surgical tag at a second time, a respective subsequent measured magnitude of the magnetic field at a respective second position of each magnetically-sensitive surgical tag;
transmitting, with the respective data transmitter, the respective subsequent measured magnitude from each magnetically-sensitive surgical tag to the computer; and
determining, with the computer and using the respective initial subsequent measured magnitudes, a respective relative first position and a respective relative second position of each magnetically-sensitive surgical tag along the axis, the respective relative first position and the respective relative second position measured with respect to the one or more magnetic field gradient coil(s).

2. The method of claim 1, further comprising:
comparing, in the computer, the respective relative first position and the respective relative second position of each magnetically-sensitive surgical tag;
when a difference between the respective relative first position and the respective relative second position is within a threshold distance, indicating, with the computer, that the respective relative first position and the respective relative second position are the same; and
when the difference between the respective relative first position and the respective relative second position is greater than the threshold distance, indicating, with the computer, that the respective relative first position and the respective relative second position are different.

3. The method of claim 1, further comprising:
comparing, in the computer, the respective relative first position and the respective relative second position of each magnetically-sensitive surgical tag;
when a difference between the respective relative first position and the respective relative second position is within a desired offset distance range, indicating, with the computer, that the respective relative first position and the respective relative second position are the same; and
when the difference between the respective relative first position and the respective relative second position is outside of the desired offset distance range, indicating, with the computer, that the respective relative first position and the respective relative second position are different.

4. The method of claim 1, wherein:
the one or more magnetic field gradient coil(s) includes a first magnetic field gradient coil that produces a first magnetic field with respect to the first axis,
the respective initial measured magnitude is a first initial measured magnitude,
the respective subsequent measured magnitude is a first subsequent measured magnitude, and
the method further comprises:
producing, with at least a second magnetic field gradient coil, a second magnetic field having a second magnetic field gradient with respect to a second axis that is orthogonal to the first axis;
measuring, with the respective magnetic sensor of each magnetically-sensitive surgical tag at a third time, a respective second initial measured magnitude of the second magnetic field at the respective first position of each magnetically-sensitive surgical tag;
transmitting, with the respective data transmitter, the respective second initial measured magnitude from each magnetically-sensitive surgical tag to the computer;
measuring, with the respective magnetic sensor of each magnetically-sensitive surgical tag at a fourth time, a respective second subsequent measured magnitude of the second magnetic field at the respective second position of each magnetically-sensitive surgical tag; and
transmitting, with the respective data transmitter, the respective second subsequent measured magnitude from each magnetically-sensitive surgical tag to the computer.

5. The method of claim 4, further comprising:
producing, with at least a third magnetic field gradient coil, a third magnetic field having a third magnetic field gradient with respect to a third axis that is orthogonal to the first and second axes;
measuring, with the respective magnetic sensor of each magnetically-sensitive surgical tag at a fifth time, a respective third initial measured magnitude of the third magnetic field at the respective first position of each magnetically-sensitive surgical tag;
transmitting, with the respective data transmitter, the respective third initial measured magnitude from each magnetically-sensitive surgical tag to the computer;
measuring, with the respective magnetic sensor of each magnetically-sensitive surgical tag at a sixth time, a respective third subsequent measured magnitude of the third magnetic field at the respective second position of each magnetically-sensitive surgical tag; and
transmitting, with the respective data transmitter, the respective third subsequent measured magnitude from each magnetically-sensitive surgical tag to the computer.

6. The method of claim 5, further comprising displaying the respective relative first, second, and third pre-surgical positions and the respective relative first, second, and third post-surgical positions of each magnetically-sensitive surgical tag on a display operatively coupled to the computer.

7. The method of claim 5, further comprising:
comparing, in the computer, a respective first three-dimensional relative position of each magnetically-sensitive surgical tag with a respective second three-dimensional relative position of each magnetically-sensitive surgical tag, wherein:
the respective first three-dimensional relative position comprises the respective relative first, second, and third initial positions of each magnetically-sensitive surgical tag, and
the respective second three-dimensional relative position comprises the respective relative first, second, and third subsequent positions of each magnetically-sensitive surgical tag;
when a difference between the respective first three-dimensional relative position and the respective second three-dimensional relative position is less than or equal to a threshold distance with respect to the first, second, or third axis, indicating, with the computer, that the respective first three-dimensional relative position and the respective second three-dimensional relative position are the same; and
when the difference between the respective first three-dimensional relative position and the respective second three-dimensional relative position is greater than the threshold distance with respect to the first, second, or third axis, indicating, with the computer, that the respective first three-dimensional relative position and the respective second three-dimensional relative position are different.

8. The method of claim 5, further comprising:
comparing, in the computer, a respective first three-dimensional relative position of each magnetically-sensitive surgical tag with a respective second three-dimensional relative position of each magnetically-sensitive surgical tag, wherein:
the respective first three-dimensional relative position comprises the respective relative first, second, and third initial positions of each magnetically-sensitive surgical tag, and
the respective second three-dimensional relative position comprises the respective relative first, second, and third subsequent positions of each magnetically-sensitive surgical tag;
when a straight-line distance between the respective first three-dimensional relative position and the respective second three-dimensional relative position is less than or equal to a threshold distance, indicating, with the computer, that the respective first three-dimensional relative position and the respective second three-dimensional relative position are the same; and
when the straight-line distance between the respective first three-dimensional relative position and the respective second three-dimensional relative position is greater than the threshold distance, indicating, with the computer, that the respective first three-dimensional relative position and the respective second three-dimensional relative position are different.

9. The method of claim 8, further comprising:
adjusting the respective second three-dimensional relative position of at least one of the magnetically-sensitive surgical tags;
after adjusting the respective second three-dimensional relative position of at least one of the magnetically-sensitive surgical tags, sequentially producing the first, second, and third magnetic fields, the first, second, and third magnetic fields having the first, second, and third magnetic field gradients, respectively;
measuring, with the respective magnetic sensor of each magnetically-sensitive surgical tag, first, second, and third post-adjustment measured magnitudes of the first, second, and third magnetic fields, respectively, at a respective post-adjustment position of each magnetically-sensitive surgical tag;
transmitting, with the respective data transmitter, the first, second, and third post-adjustment measured magnitudes from each magnetically-sensitive surgical tag to the computer; and
determining, in the computer, a post-adjustment three-dimensional relative position of each magnetically-sensitive surgical tag based on the first, second, and third post-adjustment measured magnitudes.

10. The method of claim 9, wherein:
the straight-line distance is an initial straight-line distance, and
the method further comprises:
when a subsequent straight-line distance between the respective first three-dimensional relative position and the post-adjustment three-dimensional relative position is less than or equal to the threshold distance, indicating, with the computer, that the respective first three-dimensional relative position and the post-adjustment three-dimensional relative position are the same; and
when the straight-line distance between the respective first three-dimensional relative position and the post-adjustment three-dimensional relative position is greater than the threshold distance, indicating, with the computer, that the respective first three-dimensional relative position and the post-adjustment three-dimensional relative position are different.

11. The method of claim 1, further comprising performing one or more surgical steps between the first time and the second time.

12. The method of claim 11, wherein the one or more surgical steps comprises at least a portion of a total hip replacement surgery.

13. The method of claim 1, further comprising, with each magnetically-sensitive surgical tag, wirelessly transmitting the respective initial measured magnitude and the respective subsequent measured magnitude from the respective magnetic sensor to the respective data transmitter.

14. The method of claim 1, wherein:
the data transmitter comprises:
an LC circuit; and
a power and data management unit (PDMU) electrically coupled to the LC circuit and to the magnetic sensor, the PDMU configured to modulate an impedance of the LC circuit to transmit data to a radio-frequency (RF) coil that is operatively coupled to the computer, and the method further comprises:
generating an RF electromagnetic field with the RF coil; and
modulating the impedance of the LC circuit to transmit the respective initial measured magnitude and the respective subsequent measured magnitude.

15. The method of claim 10, further comprising wirelessly delivering power to each magnetically-sensitive surgical tag with the RF electromagnetic field.

16. The method of claim 1, wherein:
at least one of the magnetically-sensitive surgical tags is attached to a surgical screw, and
securing the at least one of the magnetically-sensitive surgical tags to an anatomical feature of the subject comprises driving the surgical screw into a bone of the subject.

17. A method for testing an anatomical fit of an acetabular cup implant, comprising:
placing the acetabular cup implant into an acetabulum of a subject, the acetabular cup implant including:
a plurality of magnetically-sensitive surgical tags to an anatomical feature of a subject, each magnetically-sensitive surgical tag including:
a respective magnetic sensor; and
a respective data transmitter in electrical communication with the respective magnetic sensor;
after placing the acetabular cup implant into the acetabulum, sequentially producing first, second, and third magnetic fields, the first, second, and third magnetic fields having first, second, and third magnetic field gradients, respectively, with respect to first, second, and third axes, respectively, that are mutually orthogonal;
measuring, with the respective magnetic sensor of each magnetically-sensitive surgical tag, first, second, and third measured magnitudes of the first, second, and third magnetic fields, respectively, at a respective position of each magnetically-sensitive surgical tag;
transmitting, with the respective data transmitter, the first, second, and third measured magnitudes from each magnetically-sensitive surgical tag to a computer;
determining, in the computer, a respective three-dimensional relative position of each magnetically-sensitive surgical tag based on the first, second, and third measured magnitudes, the respective three-dimensional relative position determined with respect to the first, second, and third magnetic field gradient coils; and
determining, in the computer, a measured theta angle and a measured femoral antevision angle of the acetabulum using the respective three-dimensional relative position of each magnetically-sensitive surgical tag.

18. The method of claim 17, further comprising:
comparing, in the computer, the measured theta angle and the measured femoral antevision angle with an ideal theta angle and an ideal femoral antevision angle, respectively; and
generating an output, with the computer, that indicates whether the measured theta angle and/or the measured femoral antevision angle is/are substantially the same as the ideal theta angle and/or the ideal femoral antevision angle, respectively.

19. A medical device comprising:
a shaft;
a magnetically-sensitive surgical tag disposed at a distal end of the shaft, the magnetically-sensitive surgical tag including:
a magnetic sensor; and
a data transmitter in electrical communication with the magnetic sensor;
a gradient-coil pad comprising a plurality of magnetic field gradient coils; and
a computer in electrical communication with the magnetically-sensitive surgical tag and with the gradient-coil pad,
wherein:
the gradient-coil pad is configured to produce a first magnetic field having a first magnetic field gradient with respect to a first axis, a second magnetic field having a second magnetic field gradient with respect to a second axis, and a third magnetic field having a third magnetic field gradient with respect to a third axis, wherein the first, second, and third axes are mutually orthogonal,
the magnetic sensor is configured to measure a magnitude of the first magnetic field, a magnitude of the second magnetic field, and a magnitude of the third magnetic field, and
the computer is configured to:
determine a relative three-dimensional position of the magnetically-sensitive surgical tag using the magnitude of the first magnetic field, the magnitude of the second magnetic field, and the magnitude of the third magnetic field, the relative three-dimensional position along each axis determined with respect to the gradient-coil pad, and
generate an output indicating the relative three-dimensional position of the magnetically-sensitive surgical tag.

20. The medical device of claim 19, wherein the first, second, and third magnetic fields are sequentially produced so as to encode the first, second, and third magnetic field gradients, respectively.

21. A medical device comprising:
a first body defining a plurality of screw holes, each screw hole configured to receive a surgical screw to attach the first body to a bone of a subject;
a second body defining an elongated cutting slot that extends parallel to a cutting axis;
a plurality of screws that adjustably attach the first and second bodies to set the cutting axis;
a first magnetically-sensitive surgical tag attached to the first body; and
a second magnetically-sensitive surgical tag attached to the second body,
wherein each of the first and second magnetically-sensitive surgical tags comprises:
a respective magnetic sensor; and
a respective data transmitter in electrical communication with the respective magnetic sensor.

22. The medical device of claim 21, further comprising:
a gradient-coil pad comprising a plurality of magnetic field coils; and
a computer in electrical communication with the first and second magnetically-sensitive surgical tags and with the gradient-coil pad,
wherein:
the gradient-coil pad is configured to produce a first magnetic field having a first magnetic field gradient with respect to a first axis, a second magnetic field having a second magnetic field gradient with respect to a second axis, and a third magnetic field having a third magnetic field gradient with respect to a third axis, wherein the first, second, and third axes are mutually orthogonal,
the respective magnetic sensor is configured to measure a magnitude of the first magnetic field, a magnitude of the second magnetic field, and a magnitude of the third magnetic field at a respective position of a respective magnetically-sensitive surgical tag, and
the computer is configured to:
determine a relative three-dimensional position of the respective magnetically-sensitive surgical tag using the magnitude of the first magnetic field, the magnitude of the second magnetic field, and the magnitude of the third magnetic field, the relative three-dimensional position along each axis determined with respect to the gradient-coil pad, and
generate an output indicating the relative three-dimensional position of the respective magnetically-sensitive surgical tag.

23. The medical device of claim 22, wherein the computer is further configured to:
repeatedly determine the relative three-dimensional position of the first magnetically-sensitive surgical tag as the bone is moved to form a virtual sphere, and
determine a mechanical axis of the bone based on the virtual sphere.

24. The medical device of claim 23, wherein the computer is further configured to determine an angle between the cutting axis and the mechanical axis using the relative three-dimensional position and a relative angle of the second magnetically-sensitive surgical tag.
